(19) United States
(12) Patent Application Publication
(10) Pub. No.: US 2026/0201035 A1
(43) Pub. Date: Jul. 16, 2026

(54) CHECKPOINT MODULATORS FOR ENHANCING IMMUNOTHERAPY

(71) Applicant: Dana-Farber Cancer Institute, Inc., Boston, MA (US)

(72) Inventors: Xin ZHOU, Boston, MA (US); Susanna ELLEDGE, Boston, MA (US); Zhixing MA, Boston, MA (US)

(73) Assignee: Dana-Farber Cancer Institute, Inc., Boston, MA (US)

(21) Appl. No.: 19/138,502

(22) PCT Filed: Dec. 13, 2023
(86) PCT No.: PCT/US2023/083836

§ 371 Date: Jun. 12, 2025

Related U.S. Application Data
(60) Provisional application No. 63/387,243, filed on Dec. 13, 2022.

(51) Int. Cl. C07K 16/28; A61K 40/11; A61K 40/31; A61K 40/42; C12N 5/0783; G01N 33/68

(52) U.S. Cl. CPC C07K 16/2818; A61K 40/11; A61K 40/31; A61K 40/421; C07K 16/2803; C12N 5/0636; G01N 33/6854; C07K 2317/31; C07K 2317/55; C07K 2317/73; C07K 2317/92; C07K 2319/02; C12N 2510/00; G01N 2440/14

(57) ABSTRACT

Disclosed are binder molecules that can bind to specific phosphorylated amino acids in proteins like signal transduction proteins and affect regulation of signal transduction pathways containing those proteins. In one example, a binder molecule can bind to an intracellular phosphotyrosine in PD-1 and prevent activation of the PD-1 pathway. Intracellular expression of these binders, in CAR-T cells for example, can prevent CAR-T cell exhaustion.

This application claims priority to U.S. Provisional Application No. 63/387,243, filed on Dec. 13, 2022, the entire contents of which are incorporated herein by reference. All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTEREST

This invention was made with government support under R00 EB030587 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### FIELD OF THE INVENTION

This invention is directed to enhancing cell therapy with a new class of immune checkpoint modulators and methods of use thereof.

### BACKGROUND

Chimeric antigen receptor (CAR)-T cell therapy has revolutionized cancer treatment within the past decade. These engineered T cells are directed towards a cancer target to destroy cancer cells expressing that target. However, like natural T cells, CAR-T cells are susceptible to exhaustion after repeated stimulation. In this case, a number of checkpoint inhibitory receptors are upregulated on the CAR-T cell surface, such as PD-1, that interact with ligand receptors on the surface of tumor cells to shut off T cell activity. While there are therapeutic antibodies that block signaling between PD-1 and cognate ligand PD-L1, patient data has shown that this is only effective in a subset of patients. Therefore, new checkpoint receptor modulators must be developed to increase the efficacy of CAR-T cells and other immunotherapies.

### SUMMARY OF THE INVENTION

Disclosed herein are immune checkpoint modulator compositions and methods of use of same.

An aspect of the invention is directed to an antibody system comprising a first antibody or antigen binding fragment thereof (B1) that binds to a phosphorylated amino acid motif in a target protein, forming a first complex of B1 and the protein, and a second antibody or antigen binding fragment thereof (B2) that binds to the first complex to form a second complex. In some embodiments, B1 can be a protein or part of a protein that can bind to a phosphorylated amino acid in the target protein. In some embodiments, B2 can be a scFv, single-domain antibody, nanobody, DARPin, affibody, and the like.

In some embodiments, the phosphorylated amino acid motif is embedded in a three-dimensional structural domain of the target protein (tertiary folded domain of the protein). In some embodiments, the phosphorylated amino acid motif that binds B1 comprises a three-dimensional (folded) epitope of the target protein. In some embodiments, the phosphorylated amino acid motif that binds BI comprises a linear epitope of the target protein.

In some embodiments, B2 bound to the first complex binds to B1. In some embodiments, B2 bound to the first complex binds to the target protein. In some embodiments, B2 bound to the first complex binds to B1 and the target protein. In some embodiments, B2 bound to the first complex recognizes an epitope comprising B1 and the target protein.

In some embodiments, B1 does not bind the amino acid motif in the target protein if the amino acid motif is not phosphorylated. In some embodiments, B2 does not bind the first complex if B1 is not bound to the phosphorylated amino acid motif in the target protein.

In some embodiments, the phosphorylated amino acid motif comprises a phosphohistidine, phosphoserine, phosphothreonine, or phosphotyrosine. In some embodiments, the phosphorylated amino acid motif can be bound by other proteins, including proteins with enzymatic activity, proteins that facilitate formation of signal-transduction complexes, and the like. In some embodiments, the phosphorylated amino acid motif comprises a phosphotyrosine to which additional proteins, for example having a phosphotyrosine-binding domain or a Src Homology 2 (SH2) domain, can bind. In some embodiments, the additional proteins can facilitate formation of signaling complexes comprising additional proteins.

In some embodiments, the phosphorylated amino acid motif is present in a signal transduction protein. In some embodiments, the signal transduction protein regulates an immune checkpoint pathway. In some embodiments, formation of the second complex prevents activation of the immune checkpoint pathway.

In some embodiments, B1 and the B2 are covalently linked.

An aspect of the invention is directed to a B1 antibody or antigen binding fragment thereof described herein. In some embodiments, B1 is covalently linked to B2.

An aspect of the invention is directed to a B2 antibody or antigen binding fragment thereof described herein. In some embodiments, B2 is covalently linked to B1.

An aspect of the invention is directed to a bispecific antibody, comprising a first antigen-binding domain (B1) that binds to a phosphorylated amino acid in a signal-transduction protein, and a second antigen-binding domain (B2) that binds to B1, and/or the signal transduction protein when B1 is bound to the phosphorylated amino acid in the signal-transduction protein.

In some embodiments, B2 binds to B1 and the signal transduction protein when B1 is bound to the phosphorylated amino acid in the signal-transduction protein. In some embodiments, the signal-transduction protein comprises an intracellular signal-transduction protein.

In some embodiments, the phosphorylated amino acid is selected from the group consisting of a serine, threonine, tyrosine and histidine. In some embodiments, the tyrosine is present in a motif that is bound by a protein having a Src Homology 2 (SH2) domain.

In some embodiments, the signal-transducing protein comprises an immune checkpoint pathway. In some embodiments, the immune checkpoint pathway protein comprises PD-1, CTLA-4, LAG-3, TIGIT, 2B4, BTLA, CD57, TIM-3, KLRG-1, or a combination thereof. In some embodiments, the signal-transduction protein comprises a PD-1 protein. In some embodiments, the phosphorylated amino acid in the PD-1 protein comprises phosphotyrosine 223 or 248.

In some embodiments, the immune checkpoint pathway is not activated when B1 binds the phosphorylated amino acid in the signal-transduction protein, and when B2 binds B1 and/or the signal-transduction protein.

In some embodiments, the phosphorylated amino acid in the signal-transduction protein is phosphorylated by a protein kinase. In some embodiments, the protein kinase comprises a serine/threonine protein kinase, a tyrosine kinase or a histidine kinase. In some embodiments, the tyrosine kinase comprises a receptor tyrosine kinase.

In some embodiments, B2 does not bind to B1 and/or to the signal-transduction protein if B1 does not bind to the phosphorylated amino acid in the signal-transduction protein.

In some embodiments, the bispecific antibody comprises a peptide linker that links B1 and B2. In some embodiments, the peptide linker is between about 3 amino acids and about 50 amino acids in length. In some embodiments, B1 is in a first bispecific antibody and B2 to which the B1 binds is in a second bispecific antibody.

An aspect of the invention is directed to a first antigen binding domain (B1) of the bispecific antibody described herein. In some embodiments, B1 is not covalently linked to B2.

An aspect of the invention is directed to a second antigen binding domain (B2) of the bispecific antibody described herein. In some embodiments, B2 is not covalently linked to B1.

An aspect of the invention is directed to a nucleic acid encoding the isolated bispecific antibody described herein. An aspect of the invention is directed to a cell comprising the nucleic acid described herein. The cell comprising the nucleic acid can express the bispecific antibody described herein. In some embodiments, the cell comprising the nucleic acid comprises a T cell. In some embodiments, the T cell comprises a chimeric antigen receptor (CAR)-T cell.

An aspect of the invention is directed to introducing a B1-B2 molecule (e.g., a bispecific antibody) into a cell. In some embodiments, the B1-B2 molecule can be introduced using a lipid nanoparticle, can be fused to a cell-penetrating peptide, and the like.

An aspect of the invention is directed to a method for treating a cancer, comprising administering to a patient, a CAR-T cell that expresses intracellularly, the bispecific antibody described herein. In some embodiments, the bispecific antibody further comprises a phosphatase enzyme. In some embodiments, the bispecific antibody, when expressed in a CAR-T cell, can suppress activity of the CAR-T cell. In some embodiments, the bispecific antibody further comprises a kinase enzyme. In some embodiments, the bispecific antibody, when expressed in a CAR-T cell, can stimulate activity of the CAR-T cell.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic of pY-mediated signaling mechanisms in CAR-T cells.

FIG. 2A-B is a representation of the pY targeting by recombinant antibody pairs (pY-TRAP) workflow for generating synthetic binders against pY modifications. Panel A provides a schematic showing the design and affinity characterization of pY-TRAP. Panel B provides a schematic showing that binding of pY-TRAP is highly specific to its target. I: hZAP70-pY²⁴⁸ TRAP, II: hUb-pY⁵⁹ TRAP, III: hRIPK2-pY⁴⁷⁴ TRAP

FIG. 3 is a schematic showing synthetic pY-mediated pathway rewires cell signaling response.

FIG. 4 is a schematic of Natural T cells versus CAR-T cells.

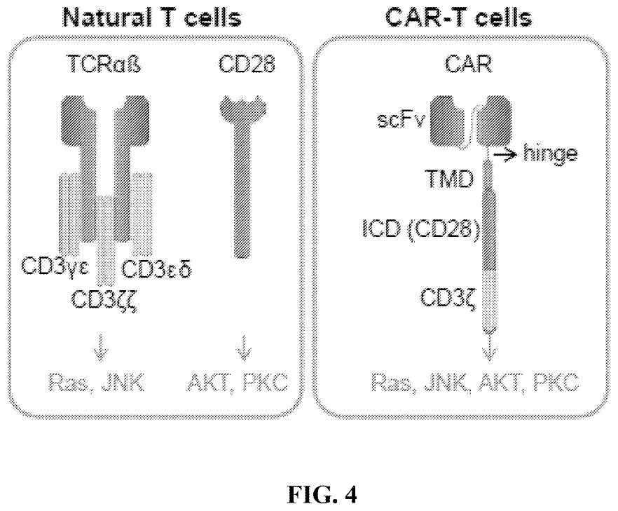

FIG. 5 is a graph showing engineered PD-1⁺ Jurkat cells.

FIG. 6A-C is a schematic showing the existing approaches to disrupt PD-1 signaling. Panel A provides a schematic of a method which includes administrating PD-1/PD-L1 blocking antibody or using an engineered CAR-T cell which secretes PD-1/PD-L1 blocking antibody. Panel B provides a schematic of a method which includes genomic disruption of PD-1, which can specifically block intrinsic PD-1 pathway in CAR-T cells. Panel C provides a schematic of a method which includes chimeric PD-1/CD28 receptors, which can invert the immunosuppressive function of the PD-1 pathway to immunostimulation.

FIG. 7A-B, Panel A provides a schematic showing B2-SH2 binders against linear pY antigens. Panel B provides a schematic showing that immune checkpoint receptors PD-1 and TIGIT both have two pY sites within the cytoplasmic signaling motifs.

FIG. 8 provides a schematic showing reprogramming the outcome of PD-1 signaling with synthetic pY binders fused to effectors.

FIG. 9 shows schematics of a natural PD-1 pathway on the left, and an example model for a synthetic PD-1 pathway on the right, where a phosphorylated amino acid in PD-1 can be targeted by a molecule as described herein (e.g., cpSH2-sdAb linked to a synthetic kinase) to affect regulation of PD-1.

FIG. 10A-B shows schematics of (A) an example pY-TRAP molecule(s) that has a B1 domain that binds a phosphorylated amino acid to form a first complex, and a B2 domain that binds the first complex, forming a second complex. (B, top) shows a schematic of an example of a pY-TRAP molecule that has a pY-binding motif from a SHP2 (B1) protein linked to an antibody (B2). (B, bottom) illustrates a schematic of an example of the region of an IgG antibody that is used as B2 in the pY-TRAP molecule.

FIG. 11A-C shows schematics (A) of a PD-1 molecule spanning the membrane of a cell and containing two intracellular phosphorylated tyrosines (pY223 and pY248). (B) shows a schematic of the SHP2 phosphatase, the effector protein that binds PD-1 in the native environment, illustrating the dual SH2 domains in SHP2 that bind the phosphorylated tyrosines in the PD-1 molecule, as shown in (A). (C) is a schematic of the circular permutated C-SH2 domain, as disclosed herein.

FIG. 11D is similar to FIG. 11A, additionally showing binding of SHP2 to the phosphorylated intracellular tyrosines in PD-1.

FIG. 12 shows example data from experiments measuring binding of various SH2 domains and the circular permutated SH2 variants to phosphorylated tyrosine in a target molecule (e.g., pY248 in PD-1) using bio-layer interferometry.

FIG. 13A-B shows schematic diagrams illustrating methods to identity B2 molecules with improved binding to the pY-BI complex using yeast display.

FIG. 14 shows example data from the experiments illustrated in FIG. 13A-B.

FIG. 15 shows example data from successive rounds of enrichment of improved B2 binders.

FIG. 16A-B shows (A) example data for binding of an individual B1-B2 clone (top) and a baseline control binder (bottom). (B) shows example data for multiple B1-B2 clones and binding to pY248.

FIG. 17 shows binding data for individual B1-B2 clones.

FIG. 18 shows example data of dose-response binding of two B1-B2 clones and a Dual SH2 control.

FIG. 19A-B shows (A) example data of three B1-B2 clones and SH2 controls binding to pY248 in PD-1 and (B) of the three B1-B2 clones binding to pY614 in the Grb1-associated binding protein 2 (Gab2). The dual SH2 domains from SHP2 non-specifically bind to both PD-1 and GAB2. The B1-B2 clone only binds to PD-1.

FIG. 20 shows example micrographs of cells in culture expressing various B1-B2 molecules.

FIG. 21 shows data from a T cell activation study using a plate-based activation assay.

FIG. 22 shows data from studies where the effects of molecules containing the CD3z domain linked to pY-TRAP molecules disclosed herein on T-cell activation were tested.

FIG. 23 shows example genetic pathways that stimulate or repress CAR-T cells.

FIG. 24 shows examples of roles played by SHP2 in T cell function.

FIG. 25 shows examples of multiple pathways regulated by SHP2 in T cells.

FIG. 26 is an example schematic illustration of immune checkpoint signaling as a contributor to CAR-T cell exhaustion.

FIG. 27 illustrates examples of reconstruction of immune cell circuits.

FIG. 28 shows data from additional studies testing dose-dependent binding of the yeast clones B #1 to B #10 to (A) pY248 in PD-1 and (B) pY614 in GAB2. Data similar to FIG. 28A are shown in FIG. 19A. Data similar to FIG. 28B are shown in FIG. 19B.

FIG. 29 shows data from studies where reconstituting PD-1 phosphorylation in HEK293 stable cells was tested. To characterize the binders in a model cell line system, PD1-TagBFP-HAtag and LCK-EGFP-v5tag were stably expressed in HEK293 cells (A, B). LCK is the kinase that phosphorylates PD-1. A PD-1-TagBFP-HAtag alone cell line was constructed as the control. As HEK cells lack endogenous PD-1 and LCK expression, PD-1 is only phosphorylated in the PD-1/LCK co-expressing cells. Membrane localization of PD-1 and LCK was confirmed by fluorescence microscopy (C). LCK dependent PD-1 phosphorylation was confirmed by immunoprecipitation experiments (D, E).

FIG. 30 provides example fluorescence images showing co-expression of binders with PD-1 and LCK in the stable HEK cells. Binders fused to mCherry in the stable HEK cells were expressed.

FIG. 31 shows data from immunoprecipitation experiments of binders #3 and 5 in cells expressing PD-1, and in cells expressing PD-1 and LCK. The binders fused to mCherry were expressed in PD-1-overexpressing or PD-1/LCK-overexpressing HEK cells. The expression of binders, PD-1, and LCK were confirmed from whole-cell lysates with anti-mCherry, anti-PD1, and anti-V5 antibodies, respectively. Immunoprecipitation (IP) experiments were performed with anti-HA beads that bind the HA-tagged PD-1-TagBFP fusion protein.

FIG. 32 provides fluorescence microscopy images showing colocalization of Binder #5 with PD-1 at the cell membrane when LCK is present. Higher magnification fluorescence images were collected for binder #5 in low and high pPD-1 cells.

FIG. 33 provides screenshots of confocal images showing Binder #5 (red) is recruited to the membrane and colocalized with PD-1 (blue) in the presence of LCK.

FIG. 34A-B shows in (A) construction of an pY-TRAP by combining a circular permuted SH2 domain (cpSH2) from SHP2 phosphatase with a synthetic nanobody library having randomized CDR sequences. (B) depicts an example of a yeast surface display screening of pY-TRAP tagged with an HA-tag to a biotinalyated peptide with phosphotyrosine modification (pY-peptide).

FIG. 34C shows an example diagram of a selection process utilizing magnetic activated and fluorescent activated cell sorting.

FIG. 34D shows an example series of clone enrichment. Each plot shows binding of pY-peptide visualized by a strepatividin-647 signal and an HA-488 signal. The populations in the upper-quadrants of the flow graphs of the plots were enriched. A titration after final round of selection showed a nanomolar range EC₅₀.

FIG. 35A-B shows in (A) titration of some enriched binders from yeast selection showed nanomolar range EC₅₀. These binders generally bound better than cpSH2 and tandemSH2 from SHP2. (B) shows example studies where specificities of selected binders were tested against a panel of peptides, including unphosphorylated forms of the peptides, and pY-peptides with similar sequences to PD-pY248 and the endogenous binding site of the SHP2 SH2 domain.

FIG. 36A-B shows in (A) depiction of a split-luciferase system where wild-type PDI(YY) or alanine mutations of both tyrosine sites (Y→A) was fused with LgBit from a luciferase. pY-TRAP binder was fused with SmBit from the luciferase enzyme. The interaction of the Binder-SmBit and PD1-LgBit reconstitute a full luciferase. (B) shows a heatmap of luciferase assay with cpSH2, TandemSH2 domain from SHP2, SHP2, and selected binders in LentiX cells expressing WT PD1 (YY) and mutant PD1 (AA). Fold change in luciferase signal is shown.

FIG. 37 shows data from example studies where cell lysates from LentiX cells coexpressing Lck kinase, PD1 WT (YY) or Y223F/Y248F (FF), and selected binders tagged with mCherry and HA were immunoprecipitated with anti-HA beads and subsequently blotted with anti-pTyr antibody. The bands show tyrosine phosphorylation with a size corresponding to PD1 and only in YY cells. Expression of Lck was detected with V5 antibody and PD1 levels were confirmed with anti-PD1 antibody in whole cell lysates.

FIG. 38 shows example cell fluorescence data of expression of GFP-tagged PD1 with either or both tyrosine residues mutated to phenylalanine in LentiX cells co-expressing Lck and mCherry tagged Binder-NT7. Membrane localization of the binder was observed in wildtype PD1 (Y223/Y248) and F223/Y248 PD1. Membrane localization of the binder was diminished in both F223/F248 PD1 and Y223/F248 PD1.

FIG. 39A shows co-culture of Raji B cells that overexpress PDL1, and Jurkat T PD1−/− cells reconstituted with wildtype PD1 (YY) or FF mutant PD1 (FF). Jurkat T cells were activated with 1000 μM anti-CD3/anti-CD19 bispecific antibody (BiTE).

FIG. 39B-C shows that, after activation of the cells shown in FIG. 39A, activation of T cells was quantified by surface CD69 levels (B) and IFN-γ release (C). The data showed that binder NT7 expression rescued T cell activation in PD1-YY mutant cells and synergized with Nivolumab (anti-PD1). In B and C, the data are means+s.d. from n=2 biological replicates. *P<0.05; **P<0.001.

FIG. 40A depicts an example synthetic transcription factor release system activated by a protease fused to a pY-TRAP molecule.

FIG. 40B shows example data from LentiX cells co-transfected with PD1-TEVsubstrate-eGFP-tTA or CD28TM-TEVsubstrate-eGFP-tTA (negative control), and BinderNT7-TEV-mCherry. In cells transfected with PD1-TEVsubstrate-eGFP-tTA, membrane recruitment of binder fused TEV was observed and subsequent nuclear localization of eGFP tagged tTA. No membrane recruitment of binder fused TEV in CD28TM-TEVsubstrate-eGFP-1TA cells was found, and no nuclear localization of tTA could be found.

FIG. 41A-B shows (A) data from biolayer interferometry (BLI) indicating that binder NT7 binds to PD-1 pY248 peptide with a K_{D} of about 17 nm. BLI data (B) also show specificity of NT7 binding to a PD-1 pY248 peptide, compared to other peptides.

### DETAILED DESCRIPTION

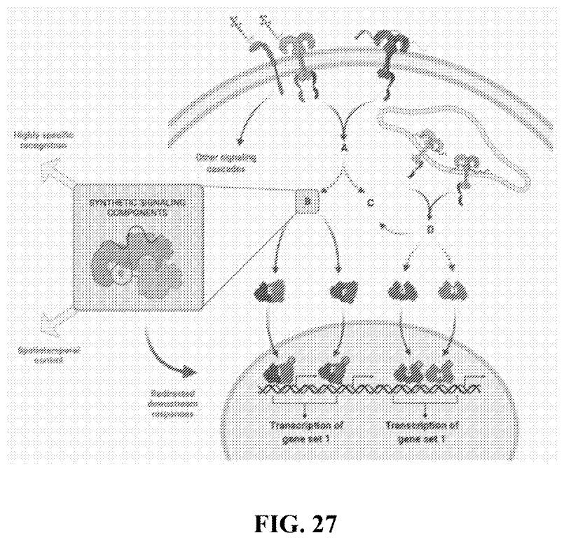

This application discloses reagents and methods for affecting regulatory pathways in cells and, in some embodiments, for creating regulatory pathways or re-wiring existing regulatory pathways in cells (FIG. 27). In some embodiments, the reagents and methods can target pathways regulated by phosphorylation and/or dephosphorylation of amino acids in proteins. In some embodiments, the reagents and methods can localize molecules that can affect cellular regulation to the vicinity of phosphorylated amino acids in a cell. The reagents and methods can be used to affect regulation of many different types of cells. In some embodiments, the targeted cells can be T cells expressing a chimeric antigen receptor (CAR-T cells).

In some embodiments, disclosed herein are binder molecules and systems thereof that can bind specific phosphorylated amino acids in specific proteins. In some embodiments, the binders and systems disclosed herein can discriminate between specific phosphorylated amino acids within a protein (e.g., bind to pY248 in PD-1, but not to pY223 in PD-1). This contrasts, for example, with existing antibodies or naturally occurring protein binding domains that can bind to specific phosphorylated amino acids (e.g., phosphotyrosine), but not to the phosphorylated amino acid only in specific proteins (e.g., only to a phosphotyrosine in PD-1, but not to phosphotyrosine in other proteins). While not wishing to be bound by theory, these generalized binders that are not protein specific can recognize a specific phosphorylated amino acid but may not recognize amino acid sequences flanking the phosphorylated amino acid in the protein. While not wishing to be bound by theory, the binders disclosed herein may be able to recognize amino acid sequences flanking or proximate to the phosphorylated amino acid, providing the protein specificity of the binders. In some embodiments, the binders disclosed herein have a higher level of specificity for the molecules to which they bind as compared to known phosphorylated amino acid binders.

### Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Chimeric Antigen Receptor (CAR) T-Cell Therapies

In some embodiments, cells expressing a chimeric antigen receptor (CAR) can be targeted using the reagents and methods disclosed herein. CAR T-cell therapies redirect a patient's T-cells to kill tumor cells by the exogenous expression of a CAR on a T-cell, for example. Other cells in which CAR can be expressed include NK (CAR NK cells) and macrophages (CAR macrophages). A CAR can be a membrane spanning fusion protein that links the antigen recognition domain of an antibody to the intracellular signaling domains of the T-cell receptor and co-receptor. CAR-T cells are generally used for treating cancer in a patient.

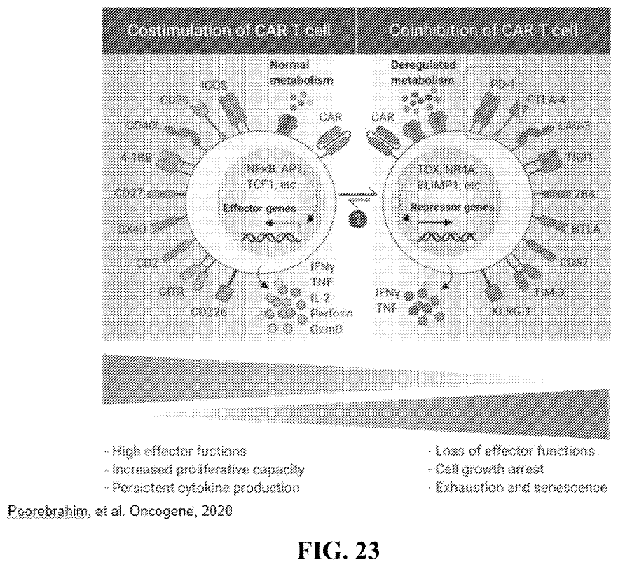

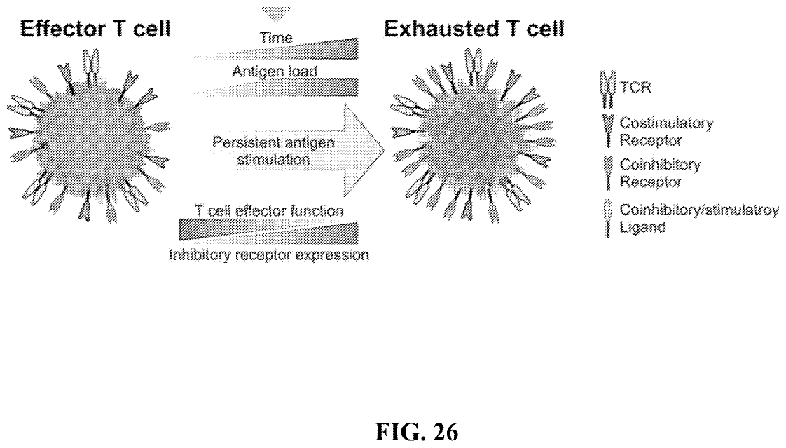

CAR-T cells can become “exhausted.” Characteristics of CAR-T exhaustion can include expression of checkpoint molecules and decreased effector functions of the cells (FIGS. 23 and 26). While not wishing to be bound by theory, it is believed that CAR-T exhaustion results from excessive or persistent antigen signaling. An immunosuppressive tumor microenvironment can contribute to this.

Solid tumors offer unique challenges for CAR-T therapies. Some barriers to CAR-T effectiveness in solid tumors include heterogeneous antigen expression, insufficient tissue homing, activation, persistence, and the immunosuppressive tumor microenvironment. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor.

In some embodiments, disclosed herein are approaches for preventing or reversing CAR-T exhaustion. In some embodiments, the disclosed approaches can be reversible (e.g., can be turned on and off).

Using the disclosed methods, for example, molecules disclosed herein can be expressed intracellularly. The molecules, in some embodiments, engineered binder systems (e.g., B1 and B2 as disclosed herein) can block functioning of certain cellular regulatory pathways, including immune checkpoint pathways. In some embodiments, the molecules disclosed herein can be used to regulate any regulatory pathway that involves phosphorylation of molecules (e.g., phosphorylation of specific amino acids in proteins). In some embodiments, the disclosed molecules can bind phosphorylated amino acids and prevent binding of other regulatory molecules. In some embodiments, the disclosed molecules can bind to phosphorylated amino acids in a protein and influence phosphorylation or dephosphorylation of molecules in the vicinity (e.g., by recruiting kinases or phosphatases to the area). In some examples, the disclosed molecules (e.g., B1 and B2) can be combined into other molecules, like kinases or phosphatases, to target those other molecules to specific cellular locations. In some examples, the disclosed molecules, when linked or tethered to a protease, intein, or a fragment of these molecules, can bring those molecules to the vicinity of a phosphorylated amino acid to which the disclosed B1/B2 molecules bind, and catalyze the release of other molecules expressing in the vicinity (e.g. transcription factors, epigenetic modulators, enzymes and the like). In some embodiments, the disclosed molecules can be minibodies, scFvs, IgG molecules, DARPins, affibodies, bispecific fusion molecules, and other antibody fragments as described herein.

The cells (e.g., CAR-T cells) expressing these molecules can be introduced into a patient in need of a treatment by infusion therapies known to one of skill in the art. In some embodiments, the patient can have a PD-1-associated disease or disorder as described herein, such as chronic infections or cancer. The cell (e.g., a T cell) can be, for instance, a T lymphocyte, a CD4+ T cell, a CD8+ T cell, or the combination thereof, without limitation. In some embodiments, the cell can be a CAR-NK cell or a CAR-macrophage cell.

In some embodiments, the molecules discussed herein can be used in the construction of multi-specific antibodies. In some embodiments, a CAR-T cell can express intracellularly, bispecific or multispecific antibodies discussed herein.

CAR-T cells can be generated according to methods known in the art using lentivirus systems (via transduction), retrovirus systems (via transfection (electroporation)), and transposon systems (via PiggyBac). The CAR-T cells used in the methods discloses herein can target different antigens. In some embodiments, the antigens can be tumor-associated antigens. Non-limiting examples of tumor-associated antigens to which CAR-T cells can target include ErbB2 (HER2/neu), carcinoembryonic antigen (CEA), epithelial cell adhesion molecule (EpCAM), epidermal growth factor receptor (EGFR), MUCI, MSLN, CD19, CD20, CD30, CD40, CD22, RAGE-1, MN-CA IX, RETI, RET2 (AS), prostate specific antigen (PSA), TAG-72, PAP, p53, Ras, prostein, PSMA, survivin, 9D7, prostate-carcinoma tumor antigen-1 (PCTA-1), GAGE, MAGE, mesothelin, β-catenin, TGF-βRII, BRCA1/2, SAP-1, HPV-E6, HPV-E7.

### Immune Checkpoint Pathways, Signal Transduction Proteins and PD-1

Immune checkpoints or immune checkpoint pathways regulate various aspects of the immune system (FIG. 23). These pathways can affect self-tolerance, immunity against cancer, and the like. In some examples, these pathways can affect T-cell activity, including CAR-T cell activity. In some embodiments, stimulators or costimulators of CAR-T cells can include ICOS, CD28, CD40L, 4-1BB, CD27, OX40, CD2, GITR, CD226 and the like. In some embodiments, inhibitors or coinhibitors of CAR-T cells can include PD-1, CTLA-4, LAG-3, TIGIT, 2B4, BTLA, CD57, TIM-3, KLRG-1 and the like. These proteins can be called signal transduction proteins. In some embodiments, these proteins can be receptors (e.g., immune checkpoint protein, cytokine receptor, and the like). In some embodiments, these proteins can have enzymatic activity. These proteins can bind with other proteins involved in signal transduction in the cell, forming what are called signal transduction pathways.

Some signal transduction proteins can contain amino acids that can be phosphorylated. In some embodiments, the phosphorylated amino acids can be located in an intracellular domain of the signal transduction protein. In some embodiments, the phosphorylated amino acids can be phosphohistidine, phosphoserine, phosphothreonine or phosphotyrosine. In some signal transduction proteins, amino acid phosphorylation can play a role in regulation of the signal transduction pathway. The phosphorylation can affect binding of various other proteins.

In some examples, various of these proteins can be part of inhibitory/coinhibitory pathways in cells and some of these pathways can be contributors to CAR-T cell exhaustion. In some embodiments, the reagents and methods disclosed herein are designed to detect or affect regulation of these pathways.

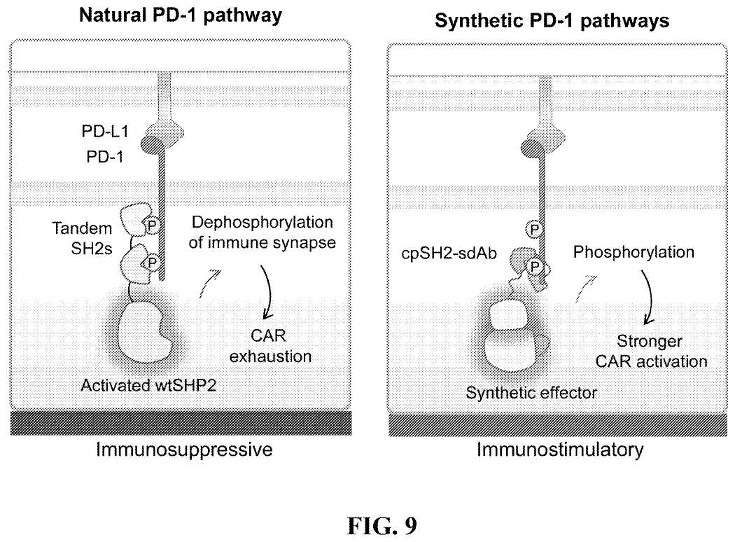

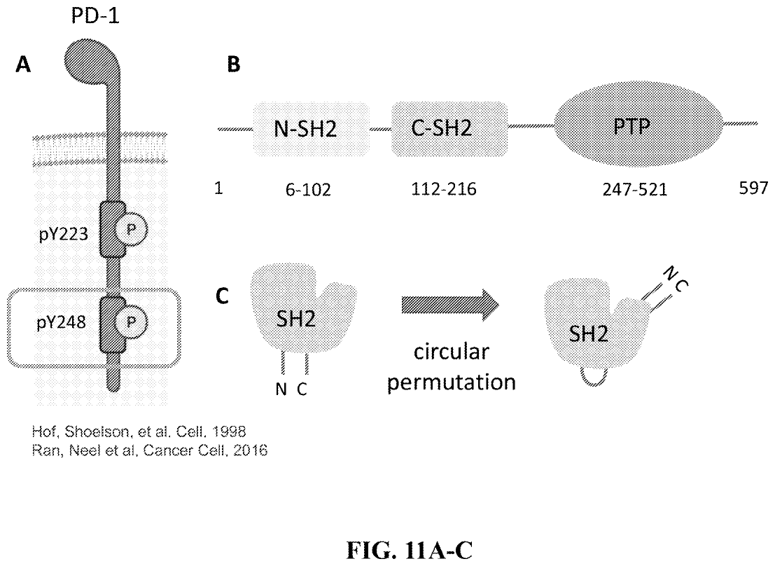

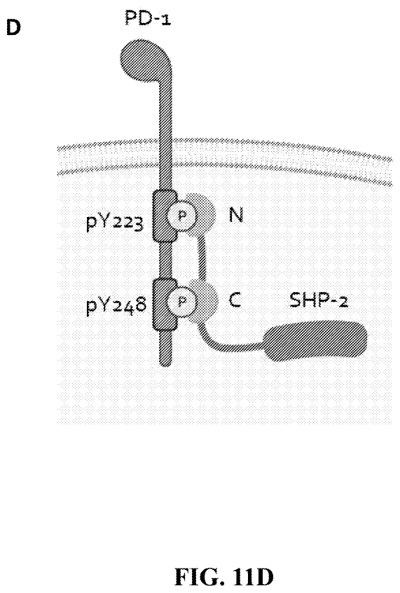

Programmed cell death-1 (PD-1), is a cell surface membrane protein of the immunoglobulin superfamily (FIGS. 9 and 11A). This protein is a signal transduction protein. This protein is expressed in pro-B-cells and is thought to play a role in their differentiation. A member of the CD28 family, PD-1 is upregulated on activated T cells, B cells, and monocytes. PD-1, as well as other immune checkpoint pathway receptors can also be upregulated in exhausted T cells. PD-1 has two identified ligands in the B7 family, PD-L1 (programmed cell death-1 ligand 1; also known as cluster of differentiation 274 (CD274) or B7 homolog 1 (B7-H1)) and PD-L2. PD-L1 is a 40 kDa type I transmembrane protein. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2. While PD-L2 expression tends to be more restricted, found primarily on activated antigen-presenting cells (APCs), PD-L1 expression is more widespread, including cells of hematopoietic lineage (including activated T cells, B cells, monocytes, dendritic cells and macrophages) and peripheral nonlymphoid tissues (including heart, skeletal, muscle, placenta, lung, kidney and liver tissues). The widespread expression of PD-L1 indicates its significant role in regulating PD-1/PD-L1-mediated peripheral tolerance. PD-1 and PD-L1 are immune down-regulators or immune checkpoint “off switches”. Examples of PD-1 inhibitors include, but are not limited to, nivolumab, (Opdivo) (BMS-936558), pembrolizumab (Keytruda), pidilizumab, AMP-224, MEDI0680 (AMP-514), PDR001, MPDL3280A, MEDI4736, BMS-936559 and MSB0010718C.

More recently, studies showed that some chronic viral infections and cancers have developed immune evasion tactics that specifically exploit the PD-1/PD-L1 axis by causing PD-1/PD-L1-mediated T cell exhaustion. Many human tumor cells and tumor-associated antigen presenting cells express high levels of PD-L1, which suggests that the tumors induce T cell exhaustion to evade anti-tumor immune responses. During chronic HIV infection, for example, HIV-specific CD8+ T cells are functionally impaired, showing a reduced capacity to produce cytokines and effector molecules as well as a diminished ability to proliferate. Studies have shown that PD-1 is highly expressed on HIV-specific CD8+ T cells of HIV infected individuals, indicating that blocking the PD-1/PD-L1 pathway can have therapeutic potential for treatment of HIV infection and AIDS patients. Taken together, agents that block the PD-1/PD-L1 pathway (either directly or indirectly) can provide a new therapeutic approach for a variety of cancers, HIV infection, and/or other diseases and conditions that are associated with T-cell exhaustion. Such agents are described herein.

Proteins that Bind Signal Transduction Proteins, Including SHP2 Binders

In some embodiments, proteins can bind to phosphorylated amino acids, including phosphorylated amino acids present in signal transduction proteins (e.g., phosphotyrosine). In some embodiments, the proteins that bind the phosphorylated amino acids can have enzymatic activity. In some embodiments, the proteins that bind the phosphorylated amino acids can facilitate formation of complexes of additional proteins, including proteins that form signaling complexes. In some embodiments, these proteins and complexes thereof can be involved in signal transduction pathways. In some embodiments, the proteins that bind phosphorylated amino acids can themselves contain phosphorylated amino acids. In some embodiments, the proteins that bind phosphorylated amino acids can contain enzymatic activity or can recruit proteins having enzymatic activity. In some embodiments, the enzymatic activity can be kinase activity or phosphatase activity.

In some embodiments, the proteins that bind the phosphorylated amino acids can have Src homology domains or its modified variants (e.g. with amino acid substitutions or circular permutation). Src homology domains can be Src Homology 2 (SH2). In some embodiments, the proteins that bind the phosphorylated amino acids can contain a phosphotyrosine-binding (PTB) domain or its modified variants (e.g. with amino acid substitutions or circular permutation). Other domains that can bind phosphorylated amino acids can include, for example, HYB domains, PH domains, including GEP100 PH, pleckstrin homology (PH)-like domains (e.g., GEP100 PH) PKCδ domains, PKCθ C2 domains, RKIP domains and the like.

SHP2 is an important signaling effector molecule for a variety of receptor tyrosine kinases (RTKs), including the receptors of platelet-derived growth factor (PDGFR), fibroblast growth factor (FGFR), and epidermal growth factor (EGFR). SHP2 is also an important signaling molecule that regulates the activation of the mitogen activated protein (MAP) kinase pathway which can lead to cell transformation, a prerequisite for the development of cancer. For example, SHP2 is involved in signaling through the Ras-mitogen-activated protein kinase, the JAK-STAT and/or the phosphoinositol 3-kinase-AKT pathways. SHP2 mediates activation of Erk1 and Erk2 (Erk1/2, Erk) MAP kinases by receptor tyrosine kinases such as ErbB1, ErbB2 and c-Met by modulating RAS activation.

SHP2 has two N-terminal Src homology 2 domains (N-SH2 and C-SH2; FIGS. 9, 11B and 11D), a catalytic domain (PTP), and a C-terminal tail. The two SH2 domains control the subcellular localization and functional regulation of SHP2. The molecule exists in an inactive conformation, inhibiting its own activity via a binding network involving residues from both the N-SH2 and PTP domains. In response to growth factor stimulation, SHP2 associates with the RTK signaling apparatus, and this induces a conformational change that results in SHP2 activation.

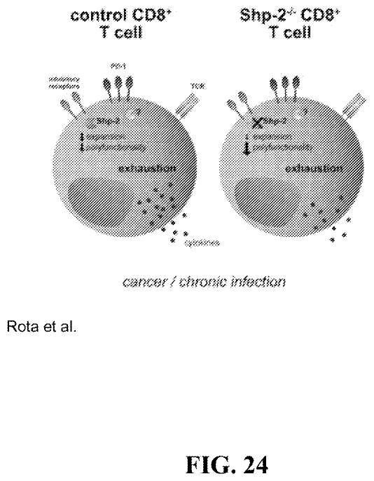

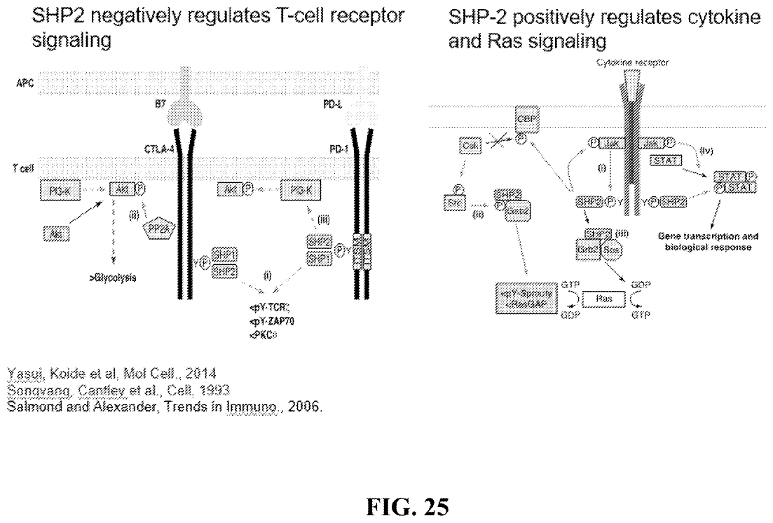

SHP2 is an immunosuppressive regulator in T cells and can play a complex role in T cell function (FIGS. 24 and 25). In some embodiments, SHP2 can play a role in CAR-T cell exhaustion. In some examples of the PD-1 regulatory pathway, binding of PDL-1 by PD-1 can cause PD-1 to be phosphorylated at pY223 and pY248 (FIG. 11A). Once phosphorylated, SHP2 can bind PD-1 at those phosphorylated amino acid residues (FIG. 11D). SHP2 has phosphatase activity and it can dephosphorylate signaling components in the vicinity. The result can be negative regulation of the T cell, including CAR-T cells.

### Binders

In the broadest sense, the term “binder” is understood to mean a molecule which binds to a target molecule. Herein, a binder molecule may be indicated by the letter “B”, which may have a following number that indicates specific binder molecules (e.g., B1, B2 and the like). The target molecule can be present in a certain target cell population. The term binder is to be understood in its broadest meaning and also comprises, for example, lectins, proteins capable of binding to certain molecules, and phospholipid-binding proteins. Such binders include, for example, high-molecular weight proteins (binding proteins), polypeptides or peptides (binding peptides), non-peptidic (e.g. aptamers (U.S. Pat. No. 5,270,163) review by Keefe A. D., et al., Nat. Rev. Drug Discov. 2010; 9:537-550), or vitamins) and other cell-binding molecules or substances. Binders can be a region or domain of one molecule that can bind to another molecule (e.g., a fragment of the first molecule that can bind to a second molecule). In embodiments, the binder can be from a signal transduction protein or a or a protein that binds a signal transduction protein. In some embodiments, the binder can bind to a phosphorylated protein or a phosphorylated amino acid motif. The binder may not bind the amino acid motif if it is not phosphorylated.

In some embodiments, a binder can be a domain of a protein that can bind to a signal transduction protein. In some embodiments, a binder can be an SH2 domain which can be from the SHP2 protein. In some embodiments, the SH2 domain can be the N-SH2 domain or the C-SH2 domain of SHP2 (FIG. 11B). In some embodiments, these SH2 domains can bind to pY223 or pY248 of PD-1 (FIG. 11D). In some embodiments, the SH2 domain does not bind to amino acid 223 or 248 in PD-1 unless the amino acid (e.g., tyrosine) is phosphorylated.

Binders can be, for example, antibodies and antibody fragments or antibody mimetics such as, for example, affibodies, adnectins, anticalins, DARPins, avimers, or nanobodies (review by Gebauer M. et al., Curr. Opinion in Chem. Biol. 2009; 13:245-255; Nuttall S. D. et al., Curr. Opinion in Pharmacology 2008; 8:608-617). Binding peptides are, for example, ligands of a ligand/receptor pair such as, for example, VEGF of the ligand/receptor pair VEGF/KDR, such as transferrin of the ligand/receptor pair transferrin/transferrin receptor or cytokine/cytokine receptor, such as TNFalpha of the ligand/receptor pair TNFalpha/TNFalpha receptor. In some embodiments, binding peptides or polypeptides can include proteins that bind to signal transduction proteins. In some embodiments, binding peptides/polypeptides can include a Src Homology 2 (SH2) domain.

In some embodiments, a binder that is an antibody can bind to a phosphorylated amino acid or a phosphorylated amino acid motif and may not bind the amino acid motif if it is not phosphorylated. In some embodiments, the phosphorylated amino acid motif can be in a signal transduction protein or a protein that binds to a signal transduction protein. In some embodiments, the antibody can bind to an amino acid motif that includes pY223 or pY248 of PD-1. In some embodiments, the antibody does not bind to an amino acid unless the amino acid is phosphorylated.

The phosphorylated amino acid motif to which an antibody binds can be an amino acid epitope. The epitope can be a linear amino acid epitope. The epitope can be embedded in a three-dimensional structural domain of a target protein. The epitope can be a non-linear amino acid epitope of the target protein. The epitope can be a three-dimensional (folded) amino acid epitope of the target protein.

The epitope may be a phosphor-serine and phosphor-threonine to which a WW domain (also called rsp5-domain or WWP domain) can bind. The epitope may be a phosphohistidine motif to which an SH2 domain can bind. The epitope may be a sulfotyrosine to which a protein having a modified SH2 domain can bind.

In some embodiments, a binder can bind to another binder that has bound to its target. In some embodiments, a first binder, B1, can target a phosphorylated amino acid motif, and forms a first complex of B1 and the phosphorylated amino acid motif. A second binder, B2, can target the first complex and forms a second complex. Herein, such antibody systems can target regulatory pathways in cells and alter their regulation or prevent their regulation from being altered. For example, formation of the second complex can prevent a molecule from binding and/or regulating a phosphorylated amino acid motif in a signal transduction molecule. Formation of the second complex can prevent activation of an immune checkpoint pathway. In some embodiments, B1 can be an antibody, ScFv, single-domain antibody, nanobody, DARPin, affibody, or the like that binds to a phosphorylated amino acid. In some embodiments, B1 can be a part of a protein that can bind to a phosphorylated amino acid. In some embodiments, B2 can be an antibody, ScFv, single-domain antibody, nanobody, DARPin, affibody, or the like that can bind to at least B1, when B1 is bound to a specific phosphorylated amino acid.

In some embodiments of the above, an SH2 domain that has bound to pY248 in PD-1 (the SH2 domain is the first binder or B1) forms a first complex between the SH2 domain and the pY248. In some embodiments, that complex can be bound by a second binder (B2) to form a second complex. In some embodiments, B2 can be an antibody. In some embodiments, B2 can bind to B1, to the target to which B1 has bound, to B1 and the B1 target, or to other configurations of epitopes. In embodiments, B2 does not bind the first complex unless B1 is bound to the B1 target protein.

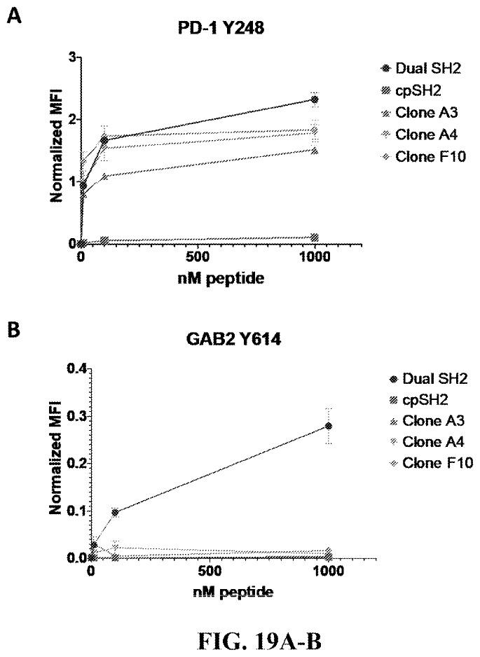

In some embodiments, a B1 binder can bind to a phosphorylated amino acid in different proteins. The B1 binders that have this property can comprise phosphorylated amino acid binding domains from proteins that can bind phosphorylated amino acids in different proteins. In some embodiments, such an amino acid binding domain is an SH2 domain from SHP2 (e.g., FIG. 25, right panel, which shows that SH2 domains in SHP2 can bind to multiple proteins, in addition to PD-1). In instances where a B1 binder can bind different phosphorylated amino acids within a protein, or can bind phosphorylated amino acids in different proteins, the B2 partner of BI can provide additional specificity. For example, data herein show that B1 binders comprising an SH2 domain from SHP2 used together with its cognate B2 binder, bind to phosphotyrosine in PD-1, but not to phosphotyrosine in GAB2 (FIGS. 19A-B).

In such antibody systems, like the B1/B2 systems described above, the B1 and B2 can be separate molecules (i.e., not covalently linked) or the B1 and B2 can be covalently linked. In some embodiments, the B1 and B2 can be linked with a peptide linker (e.g., between about 8 and about 50 amino acids in length). In some embodiments, the B1 and B2 binders can be part of a multifunctional antibody, like a bispecific antibody. The B1/B2 antibody systems can include additional functionality. For example, the B1/B2 antibody system can include a motif to which another molecule can bind. In some embodiments, the B1/B2 antibody system can include an immunoreceptor tyrosine-based activation motif (ITAM), an immunoreceptor tyrosine-based inhibitory motif (ITIM), an immunoreceptor tyrosine-based switch motif (ITSM) or the like. When expressed intracellularly, such a system would bind to specific phosphorylated amino acids in a target and would recruit kinases or phosphatases to the vicinity of the phosphorylated amino acids. The recruited kinases or phosphatases would act on substrates in the vicinity of the phosphorylated amino acids.

In some embodiments, the B1/B2 system can be integrated into enzymes, like a kinase or phosphatase enzyme. In such an embodiment, the kinase or phosphatase would bind to specific phosphorylated amino acids in a target, and the kinases or phosphates that are part of the system would act on substrates in the vicinity. In some embodiments, a B1/B2 antibody system that also contains kinase or phosphatase activity can bind a phosphorylated amino acid motif and the kinase or phosphatase is activated.

In some embodiments, the B1/B2 binder system can be fused to another enzyme. In some embodiments, the other enzyme can be a protease. In some embodiments, the B1/B2 system having a protease is expressed in a cell. A membrane-tethered effector protein (e.g., transcription factor, epigenetic modulator, enzyme, and the like) containing a substrate sequence for the protease can also be expressed in the cell. In an embodiment where B1 of the B1/B2/protease system targets a phosphorylated amino acid in PD-1, for example, the protease can be co-localized with the membrane-tethered protein, leading to cleavage of the protease substrate sequence and release of the effector protein to initiate and drive a response.

A “target molecule” in the broadest sense is understood to mean a molecule which is present in the target cell population, and which may be a protein (for example a receptor of a growth factor) or a non-peptidic molecule (for example a sugar or phospholipid). It can be a receptor or an antigen. In some embodiments, target proteins can include signal transduction proteins. In some embodiments, target proteins can include molecules like PD-1, CTLA-4, LAG-3, TIGIT, 2B4, BTLA, CD57, TIM-3, KLRG-1. In some embodiments, these molecules can be signal transduction molecules. In some embodiments, proteins can bind to signal transduction molecules and effect regulation of immune systems. In some embodiments, a target protein can have one or more phosphorylated amino acids.

The term “extracellular” target molecule describes a target molecule, attached to the cell, which is located at the outside of a cell, or the part of a target molecule which is located at the outside of a cell, i.e. a binder may bind on an intact cell to its extracellular target molecule. An extracellular target molecule may be anchored in the cell membrane or be a component of the cell membrane. The person skilled in the art is aware of methods for identifying extracellular target molecules. For proteins, this may be by determining the transmembrane domain(s) and the orientation of the protein in the membrane. These data are usually deposited in protein databases (e.g. SwissProt). In some examples, an extracellular target molecule can include a receptor or extracellular domain of a receptor.

A binder can be attached to another molecule. In some embodiments, a first binder can be attached to a second binder. In some embodiments, a first binder can be attached or linked to a second binder via a linker, which may comprise amino acids.

Attachment of the binder can be via a heteroatom of the binder. Heteroatoms according to the invention of the binder which can be used for attachment are sulphur (in one embodiment via a sulphydryl group of the binder), oxygen (according to the invention by means of a carboxyl or hydroxyl group of the binder) and nitrogen (in one embodiment via a primary or secondary amine group or amide group of the binder). These heteroatoms may be present in the natural binder or are introduced by chemical methods or methods of molecular biology. According to the invention, the attachment of the binder to the toxophore has only a minor effect on the binding activity of the binder with respect to the target molecule. In a preferred embodiment, the attachment has no effect on the binding activity of the binder with respect to the target molecule.

An “isolated” antibody, which is a type of binder, has been purified to remove other constituents of the cell. Contaminating constituents of a cell which may interfere with a diagnostic or therapeutic use are, for example, enzymes, hormones, or other peptidic or non-peptidic constituents of a cell. A preferred antibody or binder is one which has been purified to an extent of more than 95% by weight, relative to the antibody or binder (determined for example by Lowry method, UV-Vis spectroscopy or by SDS capillary gel electrophoresis). Moreover an antibody which has been purified to such an extent that it is possible to determine at least 15 amino acids of the amino terminus or of an internal amino acid sequence, or which has been purified to homogeneity, the homogeneity being determined by SDS-PAGE under reducing or non-reducing conditions (detection may be determined by means of Coomassie Blau staining or by silver coloration). However, an antibody is normally prepared by one or more purification steps.

The term “specific binding” or “binds specifically” refers to an antibody or binder which binds to a predetermined antigen/target molecule. Specific binding of an antibody or binder typically describes an antibody or binder having an affinity of at least 10-7 M (as Kd value; i.e. those with Kd values smaller than 10-7 M), with the antibody or binder having an at least two times higher affinity for the predetermined antigen/target molecule than for a non-specific antigen/target molecule (e.g. bovine serum albumin, or casein) which is not the predetermined antigen/target molecule or a closely related antigen/target molecule. The antibodies can have an affinity of at least 10-7 M (as Kd value; in other words those with smaller Kd values than 10-7 M), of at least 10-8 M, more in the range from 10-9 M to 10-11 M. The Kd values may be determined, for example, by means of surface plasmon resonance spectroscopy.

### Binders and Antibody Systems Targeting PD-1

Herein are described systems for targeting regulatory systems, including immune checkpoint pathways. In some embodiments, those systems use binders that are antibodies.

Unique recombinant monoclonal antibody clones are described herein. “Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together.

In some embodiments, a binder can be a domain from a protein that can bind to another molecule. In some embodiments, such a binder can be an SH2 domain. The SH2 domain can be from the SHP2 protein. In some embodiments, the SH2 domain from the SHP2 protein can be the C-SH2 domain or the N-SH2 that binds pY248 in PD-1 (FIGS. 11A, B and D). In some embodiments, the SH2 domain can be a circular permutated SH2 domain (FIG. 11C). In some embodiments, amino acid sequences of the circular permutated SH2 domain are shown in Tables 1 and 2.

TABLE 1
Example Nucleic Acid and Amino Acid Sequences
of Circular Permutated C-SH2
Domain from SHP2 (C-cp-SH2)

GAGCGTTTCGATTCCCTGACTGACCTTGTCGAGCACTACAAAAAGAAT
GCACCGTACTACAGTTAAAGCAACCCCTAGGTGGTTCTGGGGGGGGTT
GGTTTCATGGGCACTTGTCAGGAAAAGAGGCAGAAAAGCTGCTGACGG
AGAAAGGAAAACACGGGTCATTCTTGGTACGTGAAAGCCAGTCACACC
CCGGCGACTTTGTTCTGAGCGTAAGGACAGGTGACGATAAAGGCGAGT
CCAATGATGGGAAGAGTAAGGTTACGCATGTGATGATTCGTTGCCAGG
AGCTTAAGTACGATGTGGGCGGGGGT
(SEQ ID NO: )
ERFDSLTDLVEHYKKNPMVETLGTVLQLKQPLGGSGGGWFHGHLSGKE
AEKLLTEKGKHGSFLVRESQSHPGDFVLSVRTGDDKGESNDGKSKVTH
VMIRCQELKYDVGGG
(SEQ ID NO: )

TABLE 2
Example Nucleic Acid and Amino Acid Sequences
of Circular Permutated N-SH2
Domain from SHP2 (N-cp-SH2)

GAAAAATTTGCCACACTGGCGGAGCTGGTTCAATACTATATGGAACAT
CACGGGCAGTTAAAGGAGAAGAATGGTGACGTGATTGAACTAAAGTAC
CCGTTAGGAGGCTCAGGCGGTGGTTGGTTTCATCCCAATATCACAGGA
GTGGAAGCTGAAAATCTGTTATTAACAAGAGGCGTAGATGGCAGTTTT
CTAGCACGTCCATCCAAATCTAACCCCGGCGATTTTACCTTGAGCGTT
AGGAGGAATGGGGCAGTAACACATATTAAAATTCAAAATACCGGGGAT
TACTACGATCTTTATGGAGGG
(SEQ ID NO: )
EKFATLAELVQYYMEHHGQLKEKNGDVIELKYPLGGSGGGWFHPNITG
VEAENLLLTRGVDGSFLARPSKSNPGDFTLSVRRNGAVTHIKIQNTGD
YYDLYGG
(SEQ ID NO: )

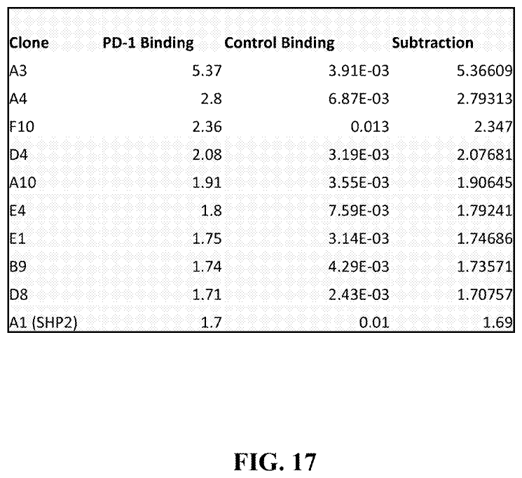

In some embodiments, the B2 antibody used in the B1/B2 binder systems described herein can be an IgG heavy chain variable region. In some embodiments, the nucleic acid sequence of the scaffold region of that region is shown in Table 3. The nucleic acid sequence scaffold of the VH domain is provided herein. In some embodiments, the VH domain can be between bases 1 and 378. In some embodiments, CDR1 can be between bases 82 and 108. In some embodiments, CDR2 can be between bases 151 and 183. In some embodiments, CDR3 can be between bases 298 and 339. In some embodiments, the CDRs of the IgG heavy chain variable region of antibodies disclosed herein are shown in Table 4 (also see Example 4, and FIG. 17).

TABLE 3
Example Nucleic Acid Sequence of
Scaffold of the VH Domain

gaggttcagctggtggagtctggcggtggcctggtgcagccagggggc
tcactccgtttgtcctgtgcagcttctggcttcgatatttatagcagc
agcgattatatcggctgggtgcgtcgcgccccgggtaagggcgaggaa
ctggttgcacgtatctatagcagcagcggctatacctattatgccgat
agcgtcaagggccgtttcactataagcgcagacacatccaaaaacaca
gcctacctacaaatgaacagottaagagctgaggacactgccgtctat
tattgtgctcgcccgttctctttctctgttgctccgtactcttacgct
attgactactggggtcaaggaaccctggtcaccgtctcctcg
(SEQ ID NO: )

TABLE 4
Example Antibody CDR Sequences: PDI pY-TRAP

| Mole- | | | |
|---|---|---|---|
| cule | V_{H} CDR1 | V_{H} CDR2 | V_{H} CDR3 |
| 10.1/ | RISSYYI | ARIYPSYGSTY | RYSFSVYPFAPSYDA |
| E4 | (SEQ ID | | |
| | NO: ) | (SEQ ID NO: ) | (SEQ ID NO: ) |
| 10.2 | DFSYSDI | ARIYPYYSYTY | RYGAM |
| | (SEQ ID | (SEQ ID NO: ) | (SEQ ID NO: ) |
| | NO: ) | | |
| 10.3/ | DIYSSSDYI | ARIYSSSGYTY | RPFSFSVAPYSYAI |
| F10 | (SEQ ID | (SEQ ID NO: ) | (SEQ ID NO: ) |
| | NO: ) | | |
| 10.4 | AIYSYDYI | ARIYPSSSYTY | RSAPAVYYVYGNGYGM |
| | (SEQ ID | (SEQ ID NO: ) | (SEQ ID NO: ) |
| | NO: ) | | |
| 10.5 | RFSSSSDI | ARIYPSSSYTY | RYAYHVYGM |
| | (SEQ ID | (SEQ ID NO: ) | (SEQ ID NO: ) |
| | NO: ) | | |
| 10.6 | AFSYSEI | ARIYPSYGYTY | RAVYYVPGYRVHAL |
| | (SEQ ID | (SEQ ID NO: ) | (SEQ ID NO:) |
| | NO: ) | | |
| 10.7 | DISSSDI | ARIYPYYSYTY | RYSAM |
| | (SEQ ID | (SEQ ID NO: ) | (SEQ ID NO: ) |
| | NO: ) | | |
| 30.3 | RISSDYI | ARIYPSYSSTY | RAYAYYVDGAL |
| | (SEQ ID | (SEQ ID NO:) | (SEQ ID NO: ) |
| | NO: ) | | |
| 30.5 | DFSSDYI | ARIYSSYG | RSYSYSVYGAAVSGL |
| | (SEQ ID | (SEQ ID NO: ) | (SEQ ID NO: ) |
| | NO: ) | | |
| 30.7 | AFSSDSI | ARIYPSYNYTS | RAVSYYVAGL |
| | (SEQ ID | (SEQ ID NO: ) | (SEQ ID NO: ) |
| | NO: ) | | |
| C6 | RIYSSSSI | ARIYPYYGSTY | RSPSPSYYYVVHGM |
| | (SEQ ID | (SEQ ID NO: ) | (SEQ ID NO: ) |
| | NO: ) | | |
| A4 | DIYSDEI | ARIYPYSSYTY | RYYYYVYGAM |
| | (SEQ ID | (SEQ ID NO: ) | (SEQ ID NO: ) |
| | NO: ) | | |
| B5 | DFYSDYI | ARISSYYNYTS | RFVYWVASPSDYYFVYSAF |
| | (SEQ ID | (SEQ ID NO: ) | (SEQ ID NO: ) |
| | NO: ) | | |
| C8 | FSYSDYI | ARIYPYYGYTY | RSGYYWVYGAM |
| | (SEQ ID | (SEQ ID NO: ) | (SEQ ID NO: ) |
| | NO: ) | | |
| F9 | RFYYSDI | ARISPYSSYTS | RYYYSVYPVRGSSGL |
| | (SEQ ID | (SEQ ID NO: ) | (SEQ ID NO: ) |
| | NO: ) | | |
| E10 | DFSSSD | ARIYPYYSYTY | RYSAM |
| | (SEQ ID | (SEQ ID NO: ) | (SEQ ID NO: ) |
| | NO: ) | | |
| D9 | DISSESI | ARISSSSSYTY | RFYYYVDGYPSYPYYAL |
| | (SEQ ID | (SEQ ID NO: ) | (SEQ ID NO: ) |
| | NO: ) | | |

In some embodiments, a scaffold for a B2 antibody used in the B1/B2 binder systems described herein can be an IgG heavy chain variable region (e.g., nanobody). In some embodiments, the amino acid sequence of the scaffold region of that region is shown in

### Table 5.

TABLE 5
Example Amino Acid Sequence of
Scaffold of the VH Domain

QLQESGGGLVQAGGSLRLSCAAS-CDR1-GWYRQAPGKER-CDR2-
ADSVKGRFTISRDNAKNTVYLQMNSLKPEDTAVYYC-CDR3-
WGQGTQVTVSS
(SEQ ID NO: )

In some embodiments, an example sequence of a pY-TRAP molecule can be as shown in Table 6.

TABLE 6
Example Amino Acid Sequence of pY-TRAP Molecule

ERFDSLTDLVEHYKKNPMVETLGTVLQLKQPLGGSGGGWFHGHLSGKE
AEKLLTEKGKHGSFLVRESQSHPGDFVLSVRTGDDKGESNDGKSKVTH
VMIRCQELKYDVGGGGGSQLQESGGGLVQAGGSLRLSCAAS-CDR1-
GWYRQAPGKER-CDR2-ADSVKGRFTISRDNAKNTVYLQMNSLKPEDT
AVYYC-CDR3-WGQGTQVTVSS
(SEQ ID NO: )

In some embodiments, the CDRs of the IgG heavy chain variable region of antibodies disclosed herein are shown in Table 7.

TABLE 7
Additional Example Antibody CDR Sequences in PDI pY-TRAP Molecules

| | | | | EC₅₀ |
|---|---|---|---|---|
| Molecule | V_{H} CDR1 | V_{H} CDR2 | V_{H} CDR3 | (nM) |
| C12 | GNIFNSDWM | ELVAGIGVGGTTNY | AAYNYGAYQESVGNTLYYSY | 1.015 |
| | (SEQ ID NO: ) | (SEQ ID NO: ) | (SEQ ID NO: ) | |
| NT7 | GYIFSRWYM | EFVAGIDQGSITYY | AAVAWAGYPWYYKHAY | 1.305 |
| | (SEQ ID NO: ) | (SEQ ID NO: ) | (SEQ ID NO: ) | |
| NT6 | GTIFRYEDM | ELVASISYGGITYY | AAVFTYGSALGY | 4.231 |
| | (SEQ ID NO: ) | (SEQ ID NO: ) | (SEQ ID NO: ) | |
| N2 | GNIFGYSYM | EFVAAIDRGGSTYY | AVLYTRGGYLGY | 4.447 |
| | (SEQ ID NO: ) | (SEQ ID NO: ) | (SEQ ID NO: ) | |
| NT9 | GSIFGGWRM | EFVATITDGSTTYY | AASLYSPVALAY | 6.042 |
| | (SEQ ID NO: ) | (SEQ ID NO: ) | (SEQ ID NO: ) | |
| NT1 | GYIFDAYNM | EFVAGINAGGITYY | AAVDDAYPPHSY | 8.34 |
| | (SEQ ID NO: ) | (SEQ ID NO: ) | (SEQ ID NO:) | |
| N1 | GYISKNPVM | ELVAGITYGTTTYY | AVGPIYPGYIDERFGYDYQY | 15.38 |
| | (SEQ ID NO: ) | (SEQ ID NO: ) | (SEQ ID NO: ) | |
| NT11 | GNIFGAYTM | EFVAGINYGTSTYY | AAVDDYSFILGSSTHLY | 16.1 |
| | (SEQ ID NO: ) | (SEQ ID NO:) | (SEQ ID NO: ) | |
| NT5 | GYIFRTDYM | EFVAAIDYGTNTNY | AAYYDYDWDRPENTYGYFRY | 20.41 |
| | (SEQ ID NO: ) | (SEQ ID NO: ) | (SEQ ID NO: ) | |
| NT3 | GYISTSLDM | EFVAAINSGATTYY | AAWSRPSYTLSY | 26.56 |
| | (SEQ ID NO: ) | (SEQ ID NO: ) | (SEQ ID NO: ) | |
| NT12 | GSISHSQYM | EFVAGIAGGTNTNY | AVPGYGRYQRTSVFKY | 40.89 |
| | (SEQ ID NO: ) | (SEQ ID NO: ) | (SEQ ID NO: ) | |
| N6 | GTIFSGWYM | EFVATIDPGGNTYY | AARYQDSHADGFTLGY | 57.05 |
| | (SEQ ID NO: ) | (SEQ ID NO: ) | (SEQ ID NO: ) | |
| NT10 | GYIFGGNIM | ELVAGINSGSNTNY | AVSYSEGGPFYY | 114.2 |
| | (SEQ ID NO: ) | (SEQ ID NO: ) | (SEQ ID NO: ) | |

The antibodies described herein comprise a first antibody or antigen binding fragment thereof (B1) that bind to a phosphorylated amino acid motif in a target protein, forming a first complex of B1 and the protein. In another embodiment, the antibodies described herein comprise a second antibody or antigen binding fragment thereof (B2) that binds to the first complex to form a second complex.

In one embodiment, B1 is a mutant of a general anti-pY antibody. In another embodiment, B2 binds to the complex of B1 and a specific pY protein. In one embodiment, the B1 and B2 antibodies have high affinity and high specificity for the phosphorylated amino acid motifs in a target protein.

Some embodiments also feature antibodies that have a specified percentage identity or similarity to the amino acid or nucleotide sequences of the anti-pY antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-pY antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-pY antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

A “protein” or “peptide” comprises a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins can contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be just a fragment of a naturally occurring protein or peptide. A protein may be naturally occurring, recombinant, or synthetic, or any combination of these.

“Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

As used herein “kinase” refers to a large class of enzymes which catalyze the transfer of the γ-phosphate from ATP to the hydroxyl group on the side chain of Ser/Thr or Tyr in proteins and peptides and are intimately involved in the control of various important cell functions, perhaps most notably: signal transduction, differentiation and proliferation. There are estimated to be about 2,000 distinct protein kinases in the human body and although each of these phosphorylate particular protein/peptide substrates, they all bind the same second substrate ATP in a highly conserved pocket. About 50% of the known oncogene products are protein tyrosine kinases PTKs and their kinase activity has been shown to lead to cell transformation.

In some embodiments, the kinase is a tyrosine kinase. As used herein “tyrosine kinase” refers to an enzyme that phosphorylates a tyrosine residue on a protein using ATP as a substrate. In some embodiments, the tyrosine kinase is a non-receptor tyrosine kinase. The mammalian nonreceptor tyrosine kinases (NRTKs) are divided into ten families: Src, Abl, Jak, Ack, Csk, Fak, Fes, Frk, Tec, and Syk.

### Antibodies

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv, VH and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (Y, u, a, 8, ¿) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (K, 2). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-HI begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the terms “nanobody” and “isolated VHH domain” can be used interchangeably and refer to camelid single-domain antibody fragments. A “nanobody” refers to the smallest antigen binding fragment or single variable domain (“VHH”) derived from naturally occurring heavy chain antibody. Nanobodies are derived from heavy chain only antibodies, seen in camelids. In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe and Lama vicugna). A nanobody with low specificity binds to multiple different epitopes (or polypeptide regions) via a single antigen binding site or binding domain, whereas a nanobody with high specificity binds to one or a few epitopes (or polypeptide regions) via a single antigen binding site or binding domain.

It should be noted that the term “nanobody,” as used herein in its broadest sense, is not limited to a specific biological source or to a specific method of preparation. For example, the nanobodies hereof can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding such a humanized VHH domain; (4) by “camelization” of a naturally occurring VH domain from any animal species, and, in particular, from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

The term “monobody” as used herein, refers to an antigen binding molecule with a heavy chain variable domain and no light chain variable domain. A monobody can bind to an antigen in the absence of light chains and typically has three CDR regions designated CDRH1, CDRH2 and CDRH3. A heavy chain IgG monobody has two heavy chain antigen binding molecules connected by a disulfide bond. The heavy chain variable domain comprises one or more CDR regions, preferably a CDRH3 region. A “VhH” or “VHH” refers to a variable domain of a heavy chain antibody such as a monobody. A “camelid monobody” or “camelid VHH” refers to a monobody or antigen binding portion thereof obtained from a source animal of the camelid family, including animals with feet with two toes and leathery soles.

The term “DARPin” (designed ankyrin repeat protein) refers to an antibody mimetic protein having high specificity and high binding affinity to a target protein, which is prepared via genetic engineering. DARPin is originated from natural ankyrin protein and has a structure comprising at least 2 ankyrin repeat motifs, for example, comprising at least 3, 4 or 5 ankyrin repeat motifs. The DARPin can have any suitable molecular weight depending on the number of repeat motifs. DARPin includes a core part that provides structure and a target binding portion that resides outside of the core and binds to a target. The structural core includes a conserved amino acid sequence and the target binding portion includes an amino acid sequence that differs depending on the target. DARPin has target specificity similar to an antibody. Thus, a new form of a bispecific chimeric protein is provided by attaching DARPin to an antibody or antibody fragment, such as an IgG (e.g., IgG1, IgG2, IgG3 or IgG4) antibody, or an scFv-Fc antibody fragment, or the like.

As used herein, the term “affibody” refers to proteins engineered to bind to target proteins or peptides with high affinity, imitating monoclonal antibodies, and are therefore a member of the family of antibody mimetics. Affibodies are composed of a three-helix bundle domain derived from the IgG-binding domain of staphylococcal protein A. The protein domain consists of a 58 amino acid sequence, with 13 randomized amino acids affording a range of affibody variants. Despite being significantly smaller than an antibody (an affibody weighs about 6 kDa while an antibody commonly weighs about 150 kDa), an affibody molecule works like an antibody since its binding site is approximately equivalent in surface area to the binding site of an antibody.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Ka represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows the cancellation of all parameters not related to affinity, and is equal to the equilibrium binding constant, KD. (See, generally, Davies et al. (1990) Annual Rev Biochem 59:439-473).

For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the anti-pY antibody, B1 and B2 can be monovalent or bivalent, and can comprise a single or double chain. Functionally, the binding affinity of the antibodies described herein is within the range of 10⁻⁵ M to 10⁻¹² M. For example, the binding affinity of the anti-pY antibody is from 10⁻⁶ M to 10⁻¹² M, from 10⁻⁷M to 10⁻¹² M, from 10⁻⁸ M to 10⁻¹² M, from 10⁻⁹M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷M to 10⁻¹⁰ M, from 10⁻⁸ M to 10⁻¹⁰ M, from 10⁻⁹ M to 10⁻¹⁰ M, from 10⁻⁵ M to 10⁻⁹M, from 10⁻⁶ M to 10⁻⁹M, from 10⁻⁷M to 10⁻⁹ M, from 10⁻⁸ M to 10⁻⁹M, from 10⁻⁵ M to 10⁻⁸ M, from 10⁻⁶ M to 10⁻⁸M, from 10⁻⁷M to 10⁻⁸ M, from 10⁻⁵ M to 10⁻⁷M, from 10⁻⁶ M to 10⁻⁷M, or from 10⁻⁵ M to 10⁻⁶ M.

A target protein, or a derivative, fragment, analog, homolog or ortholog thereof, can be utilized as an immunogen in the generation of antibodies that immunospecifically bind these protein components. A target protein or a derivative, fragment, analog, homolog, or ortholog thereof, coupled to a proteoliposome can be utilized as an immunogen in the generation of antibodies that immunospecifically bind these protein components.

Another way to determine whether a human monoclonal antibody has the specificity of a human monoclonal antibody disclosed is to pre-incubate the human monoclonal antibody of the invention with the target protein, with which it is normally reactive, and then add the human monoclonal antibody being tested to determine if the human monoclonal antibody being tested is inhibited in its ability to bind. If the human monoclonal antibody being tested is inhibited then, in all likelihood, it has the same, or functionally equivalent, epitopic specificity as the monoclonal antibody of the invention.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies, for example, are antibody molecules in which the entire sequence of both the light chain and the heavy chain, including the CDRs, arise from human genes. Such antibodies are termed “human antibodies” or “fully human antibodies”. Human monoclonal antibodies, such as fully human and humanized antibodies, can be prepared by using trioma technique; the human B-cell hybridoma technique (see Kozbor, et al, 1983 Immunol Today 4:72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from a non-human species (such as mouse), whose amino acid sequences (for example, in the CDR regions) have been modified to increase their similarity to antibody variants produced in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant can be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes the antibody of the invention or produces the antibody of the invention.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5): 489-498 (1991); Studnicka et al., Protein Engineering 7(6): 805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20).

In addition, antibodies (such as human antibodies) can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13:65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publication nos. WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein. Vectors include, but are not limited to, chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used and include moloney murine leukemia viruses. DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G., et al, Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of TIGIT in a sample. The antibody can also be used to try to bind to and disrupt a TIGIT activity.

In an embodiment, the antibodies described herein can be full-length antibodies, containing an Fc region similar to wild-type Fc regions that bind to Fc receptors.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246:1275-1281) to allow rapid and effective identification of monoclonal Fab fragments with the desired specificity for a protein or derivatives, fragments, analogs or homologs thereof. Antibody fragments that contain the idiotypes to a protein antigen can be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176:1 191-1 195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al, Anti-Cancer Drug Design, 3:219-230 (1989)). In one embodiment, the antibody of the invention has modifications of the Fc region, such that the Fc region does not bind to the Fc receptors. For example, the Fc receptor is Fcγ receptor. Antibodies with modification of the Fc region such that the Fc region does not bind to Fcγ, but still binds to neonatal Fc receptor are useful as described herein.

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn can have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is an LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In a particular embodiment, the antibody comprises an Fc variant with an amino acid substitution at amino acid position 228 or 299 (EU numbering). In more particular embodiments, the antibody comprises an IgGl or IgG4 constant region comprising an S228P and a T299A mutation (EU numbering).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238:1098 (1987). Carbon-14-labeled l-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al, Science 238:1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82:3688 (1985); Hwang et al, Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al, J. Biol. Chem., 257:286-288 (1982) via a disulfide-interchange reaction.

### Multispecific Antibodies (Bispecific and Trispecific)

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. This invention provides for multispecific antibodies, such as bi-specific and trispecific antibodies, that recognize a phosphorylated amino acid motif in a target protein and a second antigen and/or a third antigen. In one embodiment, multispecific antibodies (e.g., bi-specific antibodies and trispecific antibodies) can comprise anti-pY specific fusion proteins encompassing the antibodies described herein. Exemplary second and or third antigens include tumor associated antigens (e.g., LINGO1), cytokines (e.g., IL-12 (IL-12A (p35 subunit) protein sequence having NCBI Reference No. NP_000873.2; IL-12B (p40 subunit) protein sequence having NCBI Reference No. NP_002178.2); IL-18 (protein sequence having NCBI Reference no. NP_001553.1); IL-15 (protein sequence having NCBI Reference No. NP_000576.1); IL-7 (protein sequence having NCBI Reference No. NP_000871.1); IL-2 (protein sequence having NCBI Reference No. NP_000577.2); and IL-21 (protein sequence having NCBI Reference No. NP_068575.1)), cytokine cognate receptors (eg., IL-12R), and cell surface receptors. Non-limiting examples of second and/or third antigens include CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM (or BTLA), CD47, CD73, PD-1, 2B4, CD57, KLRG-1, or a combination thereof. In one embodiment, the bispecific antibody comprises a first antigen-binding domain (B1) and a second antigen-binding domain (B2). In one embodiment, the first antigen-binding domain (B1) binds to a phosphorylated amino acid in a signal-transduction protein. In another embodiment, the second antigen-binding domain (B2) binds to B1 and/or the signal transduction protein when B1 is bound to the phosphorylated amino acid in the signal-transduction protein.

Multispecific antibodies (e.g., bispecific antibodies and trispecific antibodies) of the invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds.

### Use of Antibodies Against PD1 (Methods of Treatment)

In various embodiments, antibodies of the invention specifically binding a phosphorylated amino acid in a signal-transduction protein, or a fragment thereof can be expressed in cells so that the antibodies can affect regulation of the cell. In some embodiments, the cells include CAR-T cells. In some embodiments, CAR-T cells expressing the antibody systems disclosed herein can be infused into a patient. In some embodiments, the patient can have cancer. In some embodiments, the cancer can be a blood cancer. In some embodiments, the cancer can be a solid cancer, including lung cancer, kidney cancer, ovarian cancer, prostate cancer, colon cancer, breast cancer, cervical cancer, uterine cancer, brain cancer, skin cancer, liver cancer, pancreatic cancer, or stomach cancer.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer (for example, if an early detection cancer biomarker is identified in such a subject), or other cell proliferation-related diseases or disorders. Such diseases or disorders include but are not limited to, e.g., those diseases or disorders associated with aberrant expression of PD-1. For example, the methods are used to treat, prevent or alleviate a symptom of cancer. In an embodiment, the methods are used to treat, prevent or alleviate a symptom of a solid tumor. Non-limiting examples of other tumors that can be treated by compositions described herein comprise lung cancer, ovarian cancer, prostate cancer, colon cancer, cervical cancer, brain cancer, skin cancer, liver cancer, pancreatic cancer or stomach cancer. Additionally, the methods of the invention can be used to treat hematologic cancers such as leukemia and lymphoma. Alternatively, the methods can be used to treat, prevent or alleviate a symptom of a cancer that has metastasized. For example, cancers that can be treated or prevented or for which symptoms can be alleviated include B-cell chronic lymphocytic leukemia (CLL), non-small-cell lung cancer, melanoma, ovarian cancer, lymphoma, or renal-cell cancer. Cancers that can also be treated or prevented or for which symptoms can be alleviated include those solid tumors with a high mutation burden and WBC in filtrate. Cancers that can be treated or prevented or for which symptoms can be alleviated further include cancers where signals in the PD-1/TIGIT axis have been modulated, cancers which include (but are not limited to) breast cancer, lung cancer (e.g., non-small cell lung cancer or lung adenocarcinoma), gastric cancer, colorectal cancer, bladder cancer, pancreatic cancer, prostate cancer, esophageal squamous cell carcinoma, nasopharyngeal carcinoma, and liquid tumors with the PD-1/PDL1 axis active (such as diffuse large B-cell lymphoma (DLBCL) and B-cell chronic lymphocytic leukemia (B-CLL)) (see e.g., Han et al., PD-1/PD-L1 pathway: current researches in cancer, Am J Cancer Res 2020; 10(3):727-742).

Accordingly, in one aspect, the invention provides methods for preventing, treating or alleviating a symptom cancer or a cell proliferative disease or disorder in a subject by administering to the subject a monoclonal antibody, scFv antibody or bi-specific antibody of the invention. For example, cells expressing the antibody systems disclosed herein can be administered in therapeutically effective amounts.

### Pharmaceutical Compositions

Cells expressing the antibodies of the invention, in some embodiments, can be administered for the treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of the cells of the invention can be the amount needed to achieve a therapeutic objective.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

### Combinatory Methods

Compositions of the invention as described herein can be administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In additional embodiments, the compositions of the invention as described herein can be administered in combination with cytokines. Cytokines that may be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

Also disclosed herein are nucleic acids encoding any of the binder molecules disclosed herein. Vectors that contain the nucleic acids encoding the binder molecules and cells that contain the vectors are contemplated. In some embodiments, the cells containing the nucleic acids and/or vectors can be T cells. The cells can be CAR-T cells, CAR-NK cells, CAR-macrophage cells and the like.

Also disclosed herein are methods for introducing the antibodies or antibody systems disclosed herein, or genes encoding the antibodies or antibody systems, into cells. In some embodiments, the antibodies/antibody systems or encoding genes can be introduced into cells in vitro or ex vivo. After introduction, the cells expressing the antibodies can be introduced into a patient. In some embodiments, the antibodies/antibody systems or genes encoding the antibodies/antibody systems can be introduced into a patient and targeted to a desired cell type, like T cells, NK cells, macrophages, and the like.

In some embodiments, the antibodies/antibody systems or genes encoding the antibodies/antibody systems can be introduced into cells using lipid nanoparticles or a viral vector. In some embodiments, lipid nanoparticles can contain mRNA encoding the antibodies/antibody systems that can be used to introduce the mRNA into cells. In some embodiments, the antibodies/antibody systems disclosed herein can be linked to a toxin or toxin fragment. The toxin/toxin fragment can be contacted with a cell. The toxin/toxin fragment can bind a receptor on a surface of the cell. The toxin/toxin fragment can be internalized by the cell, also internalizing the linked antibodies/antibody systems.

In some embodiments, the reagents and methods disclosed herein can be used as immune assay reagents or biosensors to detect the presence of specific phosphorylated amino acids in cells. As disclosed elsewhere herein, the binders can bind specific phosphorylated amino acids (e.g., phosphotyrosine), but can also bind these phosphorylated amino acids only within specific proteins (e.g., within PD-1, but not phosphotyrosine in other proteins). The binders disclosed herein can be used to detect and visualize the presence of phosphorylated amino acids in specific proteins within cells.

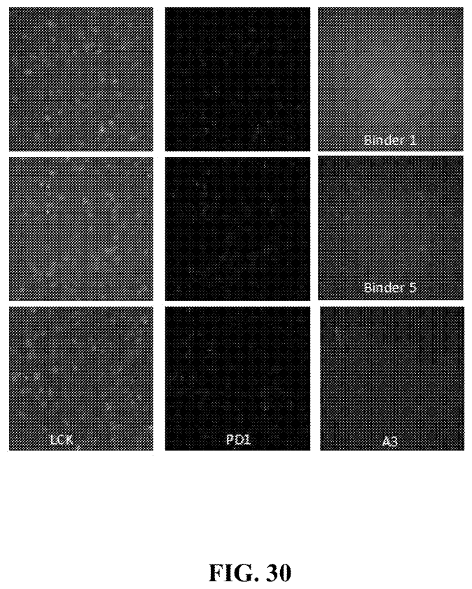

In some embodiments, the binders used as biosensors can be detected within a cell when they bind to a specific phosphorylated amino acid within a specific protein. In some embodiments, this binding can be detected using imaging methods. In some embodiments where a binder used as a biosensor, a binder molecule can be fused to a protein that can be easily visualized, like a fluorescent protein or the like. The fluorescent protein can be detected using for example, microscopy or other imaging modalities, to confirm presence or absence of specific phosphorylated amino acids within specific proteins. An example of this is illustrated in FIGS. 30, 32 and 33. Readouts other than fluorescence can be used with these binder biosensor systems.

The disclosed binder biosensors can be used diagnostically, for example. In some embodiments, these biosensors can be used to screen cells or cell lines, or to confirm phenotypes of cells or cell lines. In some embodiments, these biosensors can be used, for example, to detect disease, to confirm that specific therapies for certain diseases will be efficacious, to detect effectiveness of therapeutics, and the like. In some embodiments, the binders used as biosensors can detect functioning or defective regulatory networks in cells.

The invention is further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Development

We observed that Jurkat cells expressing SHP2 N-SH2-C-SH2 and N-SH2-C-SH2-ITAM (which bind to phosphorylated PD-1) showed enhanced jurkat activation in vitro. We also have successfully screened and validated protein binders that specifically recognize phosphorylated PD-1.

### Problem

Chimeric antigen receptor (CAR)-T cell therapy has revolutionized cancer treatment within the past decade. These engineered T cells are directed towards a cancer target to destroy cancer cells expressing that target. However, like natural T cells, CAR-T cells are susceptible to exhaustion after repeated stimulation. In this case, a number of checkpoint inhibitory receptors are upregulated on the CAR-T cell surface, such as PD-1, that interact with ligand receptors on the surface of tumor cells to shut off T cell activity. While there are therapeutic antibodies that block signaling between PD-1 and cognate ligand PD-L1, patient data has shown that this is only effective in a subset of patients. Therefore, new checkpoint receptor inhibitors would be desirable.

Our approach is to develop binders that bind to the tyrosine phosphorylated or “active” form of PD-1 inside CAR-T cells and use those binders to modulate CAR-T cell exhaustion inside of the cell. Using the pY-TRAP method previously developed by Dr. Xin Zhou (Zhou, Xin X., et al. “Targeting phosphotyrosine in native proteins with conditional, bispecific antibody traps.” Journal of the American Chemical Society 142.41 (2020): 17703-17713) and yeast surface display, we have selected binders that are a fusion of a circular permutated SH2 of SHP-2 (phosphatase that binds PD-1) and the heavy chain of a Fab antibody. This approach has many advantages over other methods to control T-cell exhaustion because we are specifically targeting the source of the exhausted signal and can rewire this signal to a number of different outputs. Additionally, we can control the expression of this binder to turn the PD-1 signaling ON/OFF which research shows may be better than just a knock-out of PD-1 or other checkpoint inhibitors. The other approach would be to knock-out SHP-2, which is the phosphatase that is thought to be responsible for binding to pY PD-1 and turning off T cell activity. However, it has also been shown that knocking out SHP-2 does not prevent exhaustion of T cells. This is most likely due to the fact that SHP-2 binds to a number of different phosphor-tyrosine proteins and by completely removing it from the cell alters the T cell function in a separate manner.

We are using this with genetic engineering of CAR-T cells to treat human cancers. The binder will be expressed intracellularly in this case and thus the construct would be included with the lentivirus that introduces the CAR-T receptor. Another approach would be to deliver the protein binder intracellularly via a lipid nanoparticle, AAV method, or cell penetrating peptide.

This approach can be combined with similar binders to other phosphorylated-tyrosine sites on other checkpoint receptors. Additionally, combining this with other CAR-T engineering approaches such as knocking out TGFbeta receptor may also increase efficacy.

### Example 2

### Specific Aims

Although Chimeric Antigen Receptor (CAR)-T cell therapy has demonstrated efficacy in treating hematologic cancers, many obstacles for widespread use of these powerful anti-cancer strategies remain. A major challenge has been the lack of robust in situ methods to determine how the structure of a CAR governs the mechanisms of T cell signaling, thus preventing the rational design of a superior CAR therapeutic. Another barrier is the noxiously immunosuppressive tumor microenvironment (TME) in solid tumors due to immunosuppressive cytokines, tumor-associated macrophages, and inhibitory checkpoints that promote severe CAR-T exhaustion. Among the various inhibitory signals in the TME, the interaction between PD-1 on T cells with PD-L1 on tumor and non-tumor cells is a major driver of CAR-T cell exhaustion. Concordantly, strategies to attenuate PD-1 signaling significantly enhance CAR-T cell efficacy. However, current methods have considerable limitations due to safety concerns, low efficiency, or compensatory signaling mechanisms.

Tyrosine phosphorylation (pY) is the central and ubiquitous component of signaling pathways in T cells, mediating both activating and suppressive signaling responses. Because of these key functions, engineering strategies to modulate pY-specific signaling events will be a powerful approach to study, engineer, and optimize CAR-T cell signaling and function. However, this strategy has never been explored before because methods to engineer specific and tight synthetic anti-pY binders have not been readily available.

In previous work, we developed a powerful platform for engineering synthetic anti-pY binders called “pY-targeting by recombinant antibody pairs”, or “pY-TRAP” (Zhou, Xin X., et al. “Targeting phosphotyrosine in native proteins with conditional, bispecific antibody traps.” Journal of the American Chemical Society 142.41 (2020): 17703-17713). We engineered two binders, B1 and B2, with B1 being the mutant of a general anti-pY binder, and B2 binding to the complex of B1 and the pY antigen. This dual binder approach addresses the challenge of phosphate recognition and also promotes affinity and specificity. This is the first in vitro method that allows engineering of specific binders against pY-modifications in three-dimensional protein structures. This work opens up the possibility of rewiring endogenous signaling pathways and engineering new cell signaling responses in CAR-T cells.

Reprogram the PD-1 signaling pathway in CAR-T cells (R00 phase). When activated on a T cell, PD-1 terminates phosphorylation of signaling proteins by recruiting SHP phosphatase to its pY signaling motifs. Synthetic PD-1 pathways can be engineered using synthetic effectors that bind pY motifs on phosphorylated PD-1. We can develop specific and tight anti-pY binders against the two cytoplasmic pY modifications on PD-1 using a derivative of the pY-TRAP method. These domains can be fused to different enzymes (phosphatase or kinase) and expressed in CAR-T cells. Their superior affinity can outcompete endogenous SHP binding to phosphorylated PD-1, resulting in a customized signaling output of the PD-1 pathway. While not wishing to be held to a theory, expressing fusions to a phosphatase can result in an immunosuppressive function, while fusion to kinase can result in an immunostimulatory function. This signaling rewiring strategy can be tested to determine if it is generalizable to other immunosuppressive pathways, such as TIGIT, and study the effect of PD-1 and TIGIT co-inhibition.

### Example 3

### Research Strategy

### Background:

Chimeric antigen receptor (CAR)-T cell therapy has shown unprecedented success for the treatment of hematological malignancies, especially for B-cell leukemia and lymphoma. However, efficacy against solid malignancies, which accounts for ninety percent of all cancer mortalities, has been very limited. Many obstacles to widespread use of these powerful anti-cancer strategies remain.

A major hurdle is the inefficient activation and limited persistence of CAR-T cells, mainly as a result of T cell exhaustion caused by immunosuppressive factors in the hostile tumor microenvironment (TME) associated with solid tumors. One mechanism that plays a major role in the suppression of CAR-T cells is the interaction between programmed cell death protein 1 (PD-1), expressed on activated or exhausted CAR-T cells, and its ligand PD-L1, presented on various tumor and non-tumor cells within the TME. Concordantly, attenuating PD-1 signaling in CAR-T cells significantly enhances CAR-T cell efficacy. More than 10 clinical trials are testing strategies to block PD-1 signaling as a means to improve CAR-T cell therapies (clinicaltrials.gov). However, current approaches have limitations, including poor safety profiles or dependence on low-efficiency primary T cell genomic engineering (detailed below). A simple, safe and efficient approach to engineer CAR-T cells that are resistant to PD-1 mediated T cell dysfunction has not yet been reported.

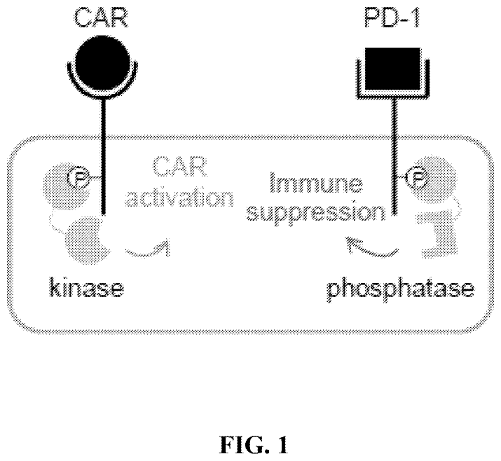

Tyrosine phosphorylation (pY) is a widespread mode of regulating protein function and underlies a critical set of signal transduction pathways in CAR-T cells (FIG. 1). During CAR activation, signaling domains such as the CD3 domain in the CAR receptor are phosphorylated in its immunoreceptor tyrosine-based activation motifs (ITAMs), which leads to the recruitment of kinases to the CAR signalosome and activation of downstream signaling pathways). In immunosuppressive pathways, a common mechanism used by many receptors (including PD-1) is to terminate phosphorylation of signaling proteins by recruiting phosphatases to their phosphorylated immunoreceptor tyrosine-based inhibitory or switch-like motifs (ITIM or ITSM, FIG. 1). Given the important functions of pY modifications in CAR-T cells, methods to modulate specific pY events represent a powerful means to study and engineer CAR-T cells. However, this approach has not been utilized, mainly because natural pY binding motifs such as Src-homology 2 (SH2) domains are highly promiscuous (i.e., the pY binding domains do not bind particular amino acids in particular proteins specifically and exclusively), and methods to engineer specific and tight synthetic anti-pY binders have not been readily available.

The studies here use a phage display platform to engineer sequence-specific anti-pY binders. The work shows how the structure of CAR can affect both stimulatory and suppressive signaling pathways. The work also addresses reprogramming of endogenous suppressive pathways to produce more powerful CAR-T cells.

Specifically, we show how to engineer a synthetic PD-1 pathway in CAR-T cells designed to reprogram the immunosuppressive function of the pathway.

### Innovation:

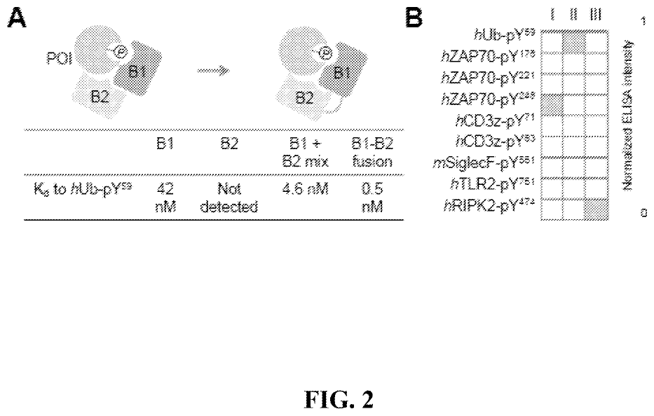

The main challenge to studying tyrosine phosphorylation is the development of specific detection and purification methods. Most antibodies to pY sequences are generated using animal immunization, but this method is low-throughput, expensive, often generates low-affinity, low-specificity, and non-renewable reagents, and is not amenable to non-immunogenic antigens. Previously, we developed an in vitro phage display workflow called “pY targeting by recombinant antibody pairs” (pY-TRAP) for generating synthetic binders against pY modifications (FIG. 2A-B; Zhou, Xin X., et al. “Targeting phosphotyrosine in native proteins with conditional, bispecific antibody traps.” Journal of the American Chemical Society 142.41 (2020): 17703-17713). In this method, we engineered two binders, B1 and B2, with B1 being the mutant of a general anti-pY antibody, and B2 binding to the complex of B1 and a specific pY protein (but not the B1 or the pY protein alone). This dual binder approach addresses the challenge of phosphate recognition and promotes enhanced affinity and sequence specificity. Linking B1 and B2 together resulted in subnanomolar Kd binders to pY modifications, which are among the tightest anti-PTM binders known to date. Herein, we describe innovative technologies based on the synthetic pY-binders to study and reprogram the action of CAR-T cells.

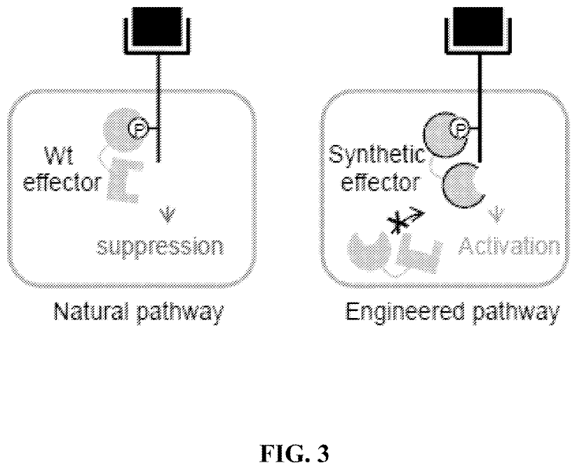

Reprograming native cell signaling pathways with a synthetic signaling output. Natural pY pathways exhibit substantial crosstalk because of the molecular promiscuity of native pY binding modalities. Such interactions typically have low affinity and modest selectivity that allows a single protein to associate with multiple partners in a dynamic manner. On the contrary, with the tight and specific nature of the pY-TRAPs, we engineer precisely linear synthetic pathways. Such synthetic pathways can allow the rewiring of endogenous signaling to drive new cell signaling responses in CAR-T cells (FIG. 3). In this work, we use such a strategy to rewire the output of PD-1 signaling to enhance the resistance of CAR-T cells to the suppressive TME (tumor microenvironment).

Although many CAR-T engineering approaches have been developed, engineering pY-binder based signaling pathway to improve CAR-T cell function has not been explored. Generalization of this approach, which is to engineer a compact synthetic signaling pathway that detects a pY signal and then rewires it to a customizable response, will allow for the construction of synthetic signaling machineries that perform a user-defined function in any biological system of interest.

### Reprogram PD-1 Signaling Pathway in CAR-T Cells.

In some embodiments, building a synthetic PD-1 pathway in CAR-T cells, in which phosphorylated PD-1 recruits synthetic effectors instead of endogenous SHP phosphatases, can rewire the output of the pathway from immunosuppression to a customized response.

### Introduction:

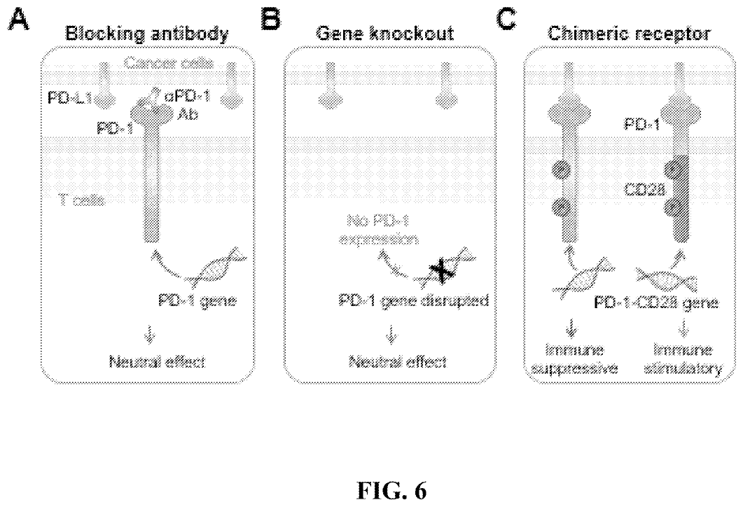

To overcome the immunosuppressive function of PD-1, methods to disrupt PD-1 signaling in CAR-T cells are being developed and have shown promise in preclinical and clinical trials. The methods include administrating PD-1/PD-L1 blocking antibody or using an engineered CAR-T cell which secretes PD-1/PD-L1 blocking antibody (FIG. 6A). However, these methods may cause a severe autoimmune response associated with systemic immune checkpoint blockade5. Alternatively, genomic disruption of PD-1 can specifically block intrinsic PD-1 pathway in CAR-T cells, but this method requires highly inefficient genome engineering of primary T cells (FIG. 6B). Finally, chimeric PD-1/CD28 receptors can invert the immunosuppressive function of the PD-1 pathway to immunostimulation, but it does not prevent the endogenous PD-1 signaling pathway from performing its immunosuppressive role (FIG. 6C).

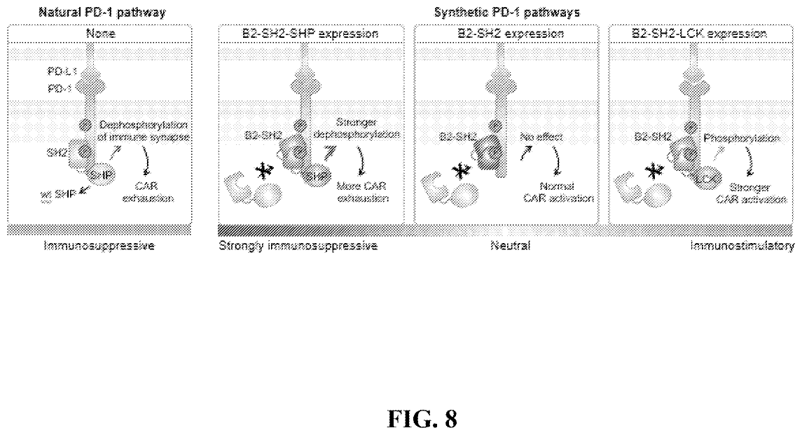

We can rewire the signaling output of the PD-1 pathway to transform it into an enhancer, rather than a suppressor of T cell activity (FIG. 8). This approach has several advantages over existing methods: (1) it modulates only the intrinsic PD-1 pathway of CAR-T cell and thus will not result in toxicity caused by systemic PD-1 blocking, (2) it does not require precise genomic editing of T cells, (3) it blocks the immunosuppressive function of the endogenous PD-1 pathway, and (4) the output can be flexibly programmed.

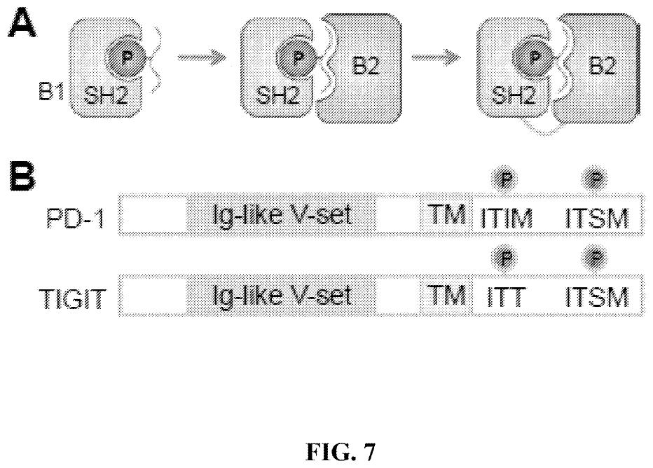

A derivative of the pY-TRAP method to engineer a “superbinder” against pY modifications in PD-1. For studying and modulating cell signaling pathways, a method to engineer binders that interact with linear pY antigens is needed. Not only are pYs in flexible linear structural motifs such as ITAM, ITIM and ITSMs widespread mediators of cell signaling transduction, but frequently used assays such as western blot require reagents that bind linear or denatured epitopes. Although the pY-TRAP method (FIG. 2) is broadly useful for pY sites in folded proteins, this approach may not be effective against linear epitopes because B1 is not optimal for peptide binding. Replacing BI in the pY-TRAP system with SH2 domains, which are natural motifs that bind to linear pY epitopes, can result in a new binding modality that recognizes linear pY antigens (FIG. 7A).

The cytoplasmic domain of PDI contains an ITIM and an ITSM motif (FIG. 7B), which are linear pY-containing motifs important for regulating immune cell signaling activities. In the endogenous PD-1 pathway, activated PD-1 recruits SHP phosphatases via interaction between the phosphorylated ITIM and ITAM and the SH2 domains in SHP phosphatases. To engineer the “superbinder” against pY sites in PD-1, we can use a SH2 domain of SHP as B1, then screen for a domain which recognizes the SH2-pY complex from our in-house Fab or single-domain VH library and use it as B2 (FIG. 7A). The library can be a cpSH2 fused to a single-domain antibody library (the VH fragment) (see Bracken, Colton J., et al. “Bi-paratopic and multivalent VH domains block ACE2 binding and neutralize SARS-CoV-2.” Nature chemical biology 17.1 (2021): 113-121). This library can be a cpSH2 fused to a nanobody library (see McMahon, Conor, et al. “Yeast surface display platform for rapid discovery of conformationally selective nanobodies.” Nature structural & molecular biology 25.3 (2018): 289-296). This B2-SH2 fusion should have much enhanced affinity against pY modifications in PD-1 than the endogenous SHP phosphatase.

Specifically, we can use two approaches to identify B2. Based on our experience developing pY-TRAP, a stable interaction (Kd<50 nM) between pY antigen/B1 is key to the successful identification of B2. Interactions between SH2 domains and pY antigens typically have a Kd of 100 nM to 10 μM and therefore requires additional engineering steps to promote a stronger interaction. In the first method, we can use methionine-based conjugation chemistry or subtiligase-based bioconjugation methods to ligate a synthetic PD-1 pY peptide to the SHP SH2 domain, and then use this covalent fusion to select for B2. The high local concentration of the two parts in a single polypeptide chain can significantly stabilize their interaction. We can also genetically fuse the SHP SH2 domain to a library of VH domain to make a phage library displaying the VH-SH2 fusion. We can then use immobilized PD-1 pY peptide to pull down the strongest interacting SH2-VH variants from this new phage library. With the obtained binders, we can engineer B2-SH2 fusions and characterize their binding affinities and specificities to the PD-1 pY antigen using biolayer interferometry. These fusions can bind to PD-1 with a much higher affinity than the SH2 domain alone in SHP. In the pY-TRAP approach, we found the B1-B2 fusions have a subnanomolar Kd to the pY antigen while either B1 or B2 bind with a Kd>10 nM (FIG. 2A). The binding to endogenous PD-1 will be validated by western blot or immunoprecipitation experiments.

Engineer synthetic PD-1 pathways in CAR-T cells. The B2-SH2 domains can be fused to different effector domains including phosphatases and kinases to create synthetic effector domains for expression in CAR-T cells (FIG. 8). While not wishing to be held to a theory, B2-SH2 fused to no effectors can inhibit PD-1 signaling, as it will bind to PD-1 with a much higher affinity than native SHP, thus preventing the recruitment of endogenous SHP to the immune synapse and its subsequent immunosuppressive function. While not wishing to be held to a theory, fusion of B2-SH2 to the phosphatase domain of SHP (B2-SH2-SHP) can be strongly immunosuppressive, because it will be more stably associated with the immune synapse than native SHP. While not wishing to be held to a theory, fusion of B2-SH2 to LCK (B2-SH2-LCK) or ZAP70 (B2-SH2-ZAP70), kinases that promotes the assembly and function of the CAR signalosome, can convert the originally immunosuppressive outcome of PD-1 signaling into a stimulatory signal.

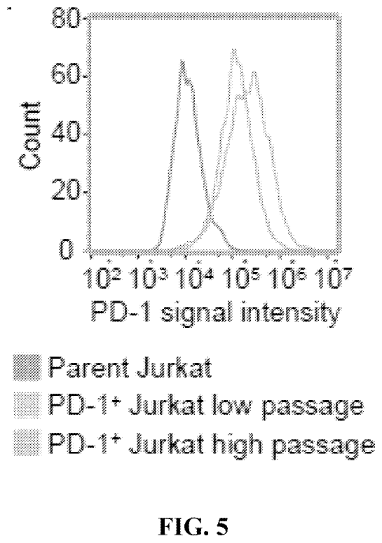

A model cell line, the PD-1+ Jurkat cell line which has homogeneous and high PD-1 expression (FIG. 5), will be used. The Jurkat cells can be infected with retroviruses encoding an anti-CD19-CAR and the synthetic effectors, and then co-cultured with CD19+PD-L1+K562 cells (described elsewhere), which can activate CAR signaling as well as the PD-L1/PD-1 pathway. We can determine if overexpression of B2-SH2, B2-SH2-SHP2, or B2-SH2-LCK in this Jurkat cell line can result in a diverse range of magnitudes of CAR activation, assayed by determining the intensity of NFAT-GFP and expression of CD69, an early surface marker of Jurkat cell activation. While not wishing to be held to a theory, B2-SH2-SHP2 expression can lead to the weakest CAR-T activation while B2-SH2-LCK/ZAP70 expression can lead to the strongest. Different orientations of protein fusions, linker lengths and mutations in the effector domains can be tested to determine the construct with a desirable signaling output.

The optimal constructs can be expressed in primary CAR-T cells. To induce an exhaustion/hypofunction phenotype, we can culture transduced T cells in the presence of 10 ng/ml Staphylococcal enterotoxin B for three days (Charles River), which can significantly increase the expression of PD-1 on the T cells. The level of PD-1 can be assayed using flow cytometry with anti-PD-1 antibody. These exhausted, PD-1+ primary CAR-T cells can be co-cultured with CD19+PD-L1+K562 cells to activate the CAR and the PD-1 signaling pathways. Surface markers (CD69/CD25/CD137), protein phosphorylation (PLC-yl-pY783, CD3-pY142, ZAP70-pY319), cytokine release (IL-2, IFN-γ, TNF-α) and cytotoxicity can be characterized to determine if cells transduced with B2-SH2 and B2-SH2-LCK elicit stronger activation/cytotoxicity than cells expressing a mock construct or B2-SH2-SHP29. These experiments can together determine if the synthetic pathway has successfully rewired the endogenous function of PD-1 and result in an impact on CAR-T cell functions.

Strategies for other immunosuppressive pathways. A comparative analysis of gene expression in patients showed that 156 genes related to immune function were differentially expressed. Despite PD-1 being a promising candidate that is being pursued on multiple fronts, these data highlight the need to consider other immune regulators. One of the analogous immunosuppressive receptors is T cell immunoglobulin and ITIM domain (TIGIT). A recent work showed overexpression of a TIGIT-CD28 chimeric receptor in CAR-T cells counteracted the immunosuppressive function of the pathway. TIGIT has a cytoplasmic tail that contains an ITIM motif and an Ig tail-tyrosine (ITT)-like motif (FIG. 7B). In the TIGIT pathway, binding to its ligand such as CD155 and CD112 induces phosphorylation of ITIM and ITT motifs of TIGIT, resulting in binding to cytosolic adapter Grb2, which then recruits SHIP1 to terminate CAR signaling. To engineer synthetic TIGIT pathway, we can follow the same strategy as proposed above. In brief, we can develop B2-SH2 binder against phosphorylated ITIM or ITT domain of TIGIT, fuse it to SHIP1 or LCK, and express it in primary CAR-T cells. To test the effect, we can make CD155+K562 and CD155-K562 cells and co-culture them with the engineered CAR-T cells. While not wishing to be held to a theory, the expression of the B2-SH2-SHIP1 fusion can result in strong immunosuppressive function, while the B2-SH2-LCK fusion can be immunostimulatory.

In some embodiments, the combined effect of co-inhibition of PD-1 and TIGIT pathways. While not wishing to be held to a theory, co-inhibition of TIGIT and PD-1 in CAR-T cells can result in more exhaustion-resistant CAR-T cell phenotypes.

### Additional Approaches:

An additional approach is to perform SH2 affinity maturation to generate a SHP (or Grb2) SH2 mutant with stronger affinity to pY sequences in PD-1 (or TIGIT), either by incorporating known mutations in SH2 domains that increased affinity to pY antigens, or developing a SH2 library to screen for tighter binders.

### Impact

The work focuses on ex vivo interrogation and engineering of primary CAR-T cells. The performance of these engineered cells in animal models can be determined.

### Example 4

We have selected binders that are a fusion of a circular permutated SH2 of SHP-2 (phosphatase that binds PD-1) and the heavy chain variable domain (VH) of a Fab antibody to target PD-1. In this example, experiments demonstrating these binders are described.

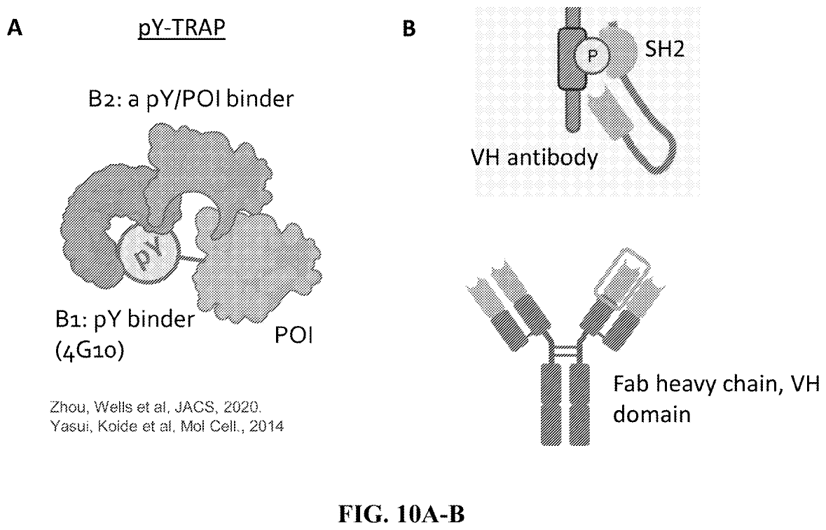

FIG. 10A illustrates pY-Trap molecules. FIG. 10B illustrates a schematic diagram of one embodiment of the molecules disclosed herein. FIG. 10B Top shows an intracellular phosphorylated tyrosine in the PD-1 molecule. SH2 designates a binder (B1) that binds to the phosphotyrosine. This binder includes an SH2 region that binds to the phosphotyrosine in PD-1. The SH2 region is linked, with a peptide linker, to a VH domain of an antibody (B2; shown in FIG. 10B Bottom). This antibody domain binds to SH2, binds to PD-1 (e.g., phosphotyrosine), or binds to SH2 and PD-1.

FIG. 11A shows the PD-1 molecule, including the extracellular and intracellular domains. The intracellular phosphorylated tyrosines pY223 and pY248 are shown. pY248 was targeted in our example system. FIG. 11B shows various domains of the protein that binds PD-1 (e.g., SHP2). The N-SH2 domain binds to pY223. The C-SH2 domain binds to pY248 A modified C-SH2 domain was used herein at the B1 binder in our molecule. FIG. 11C shows a circularly permutated version of the C-SH2 domain of the SHP2 phosphatase that was used in our molecule. This configuration of the SH2 domain facilitated its fusion to other polypeptides. FIG. 11D shows binding of SHP2 to PD-1 via the N-terminal and C-terminal SH2 domains. In addition to PD-1, the SH2 domains in SHP2 also bind other phosphorylated molecules like GAB2 pY614 (FIG. 25, right panel).

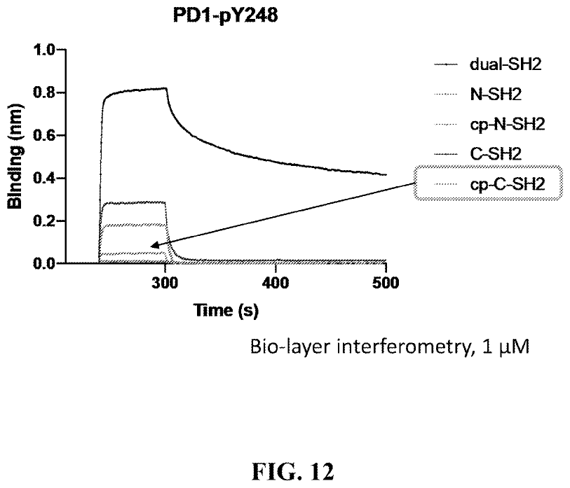

FIG. 12 shows the circular permutated version of the C-SH2 domain of the SHP2 phosphatase remained weakly binding to pY248 in the PD-1 molecule.

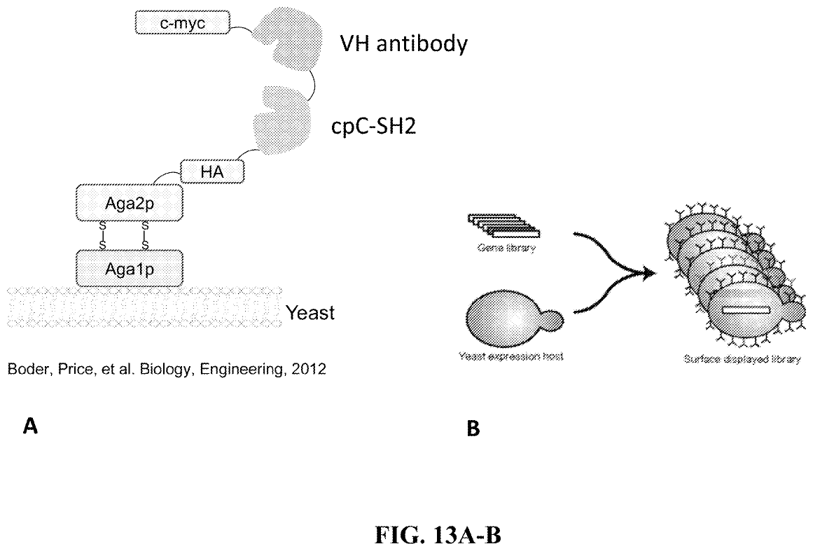

FIG. 13A-B illustrates the strategy used to isolate molecules disclosed herein. The molecules of interest were those containing the circularly permutated SH2 domain linked to a VH antibody library (also see FIG. 10B). We used the circular SH2 domain to bind to pY248 of PD-1, but the screen was designed to find antibodies from the library that, when linked to the circular SH2 domain, make the binding to pY248 stronger (e.g., better B2 antibodies). As shown in FIG. 13B, yeast display was used to express the cpC-SH2 linked to various VH antibodies from the library.

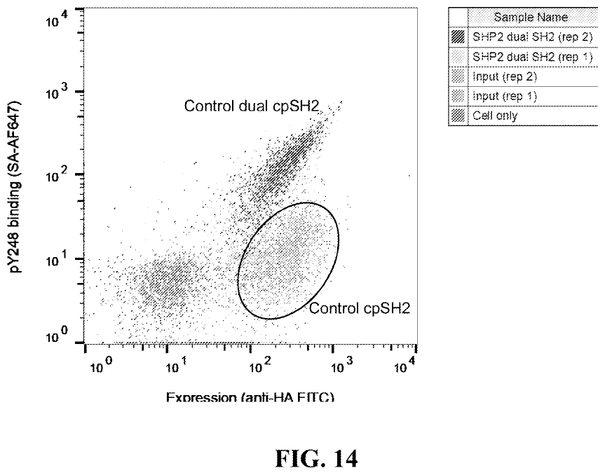

FIG. 14 shows results from screening of the yeast cells from FIG. 13B that were contacted with a peptide containing the pY248 motif from PD-1, stained and analyzed by flow cytometry. On the x-axis, yeast cells that expressed a cpC-SH2/VH antibody molecule on the cell surface were detected. On the y-axis, pY248 binding of the library of molecules expressed on the yeast cells is shown. Within the oval are yeast clones that both express the cpC-SH2/VH antibody molecules and bind pY248.

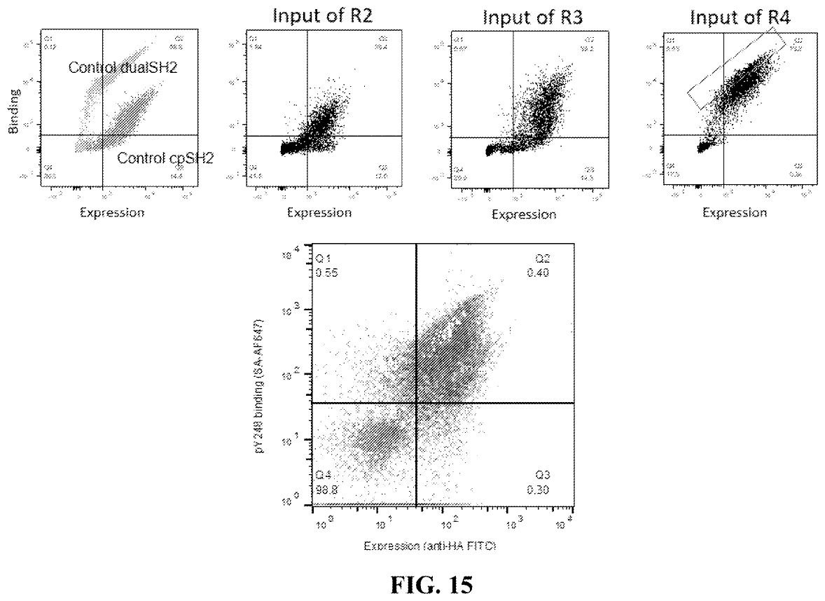

FIG. 15 shows sorting of successive rounds (R2, R³, R₄) of the cpC-SH2/VH antibody molecules (B1-B2 molecules) shown within the oval of FIG. 14 to obtain molecules that better bind pY248.

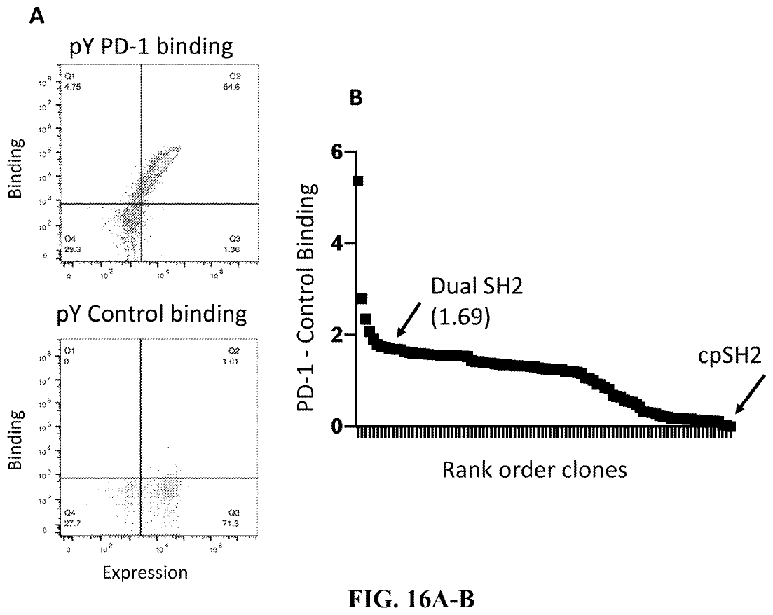

FIG. 16A-B shows individual clones obtained from cell selections from the yeast displays. FIG. 16A shows flow cytometry analysis of a negative control (bottom) and one of the high-binding clones (top). FIG. 16B shows a plot of PD-1 binding for each clone obtained from the cell sorting experiments. On the left, the Dual SH2 domains from SHP2 were the positive control for high binding. On the right, is the low binding, cpSH2 (see FIG. 12)

FIG. 17 shows binding data for 10 clones obtained from the cell sorting experiments. DNA sequences for some of these molecules were obtained. DNA sequences were also obtained for the most abundant sequences in pooled samples using next generation sequencing. CDRs contained within the VH domains from some of these molecules are shown in Table 2. The nucleic acid sequence of one of the VH regions is shown in Table 1.

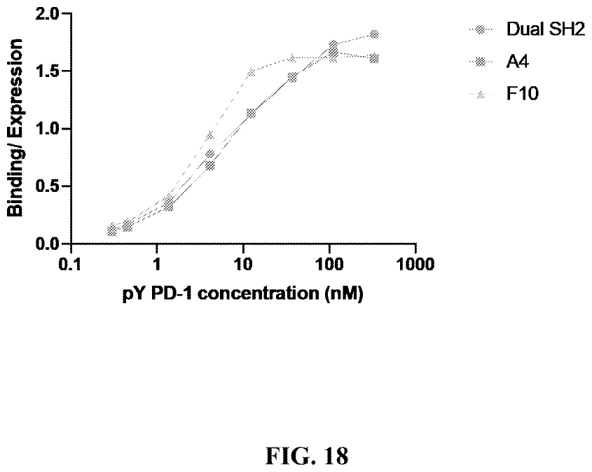

FIG. 18 shows dose-response binding curves for the A4 and F10 clones, as compared to the Dual SH2 positive control (high binder).

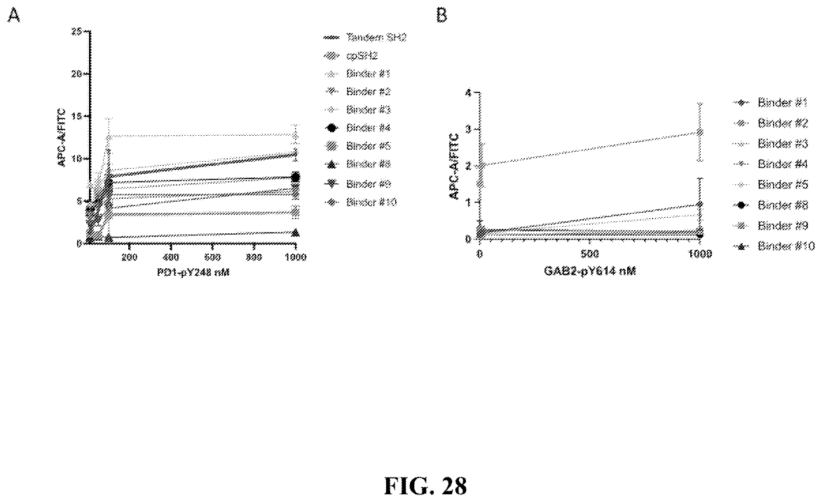

FIG. 19A shows dose-response binding curves for the A3, A4 and F10 clones, as compared to the Dual SH2 positive control (high binder) and the cpSH2 low binder to pY248 in PD-1. Additional data of this type is shown in FIG. 28A. FIG. 19B shows dose-response binding curves for the A3, A4 and F10 clones to pY614 in the Grb2-associated binding protein-2 (Gab2). Gab2 contains a SH2 domain. Gab2 is involved in granulocyte colony-stimulating factor (G-CSF) signaling. These data show the specificity of our molecules for the pY248 in PD-1. Additional data of this type is shown in FIG. 28B.

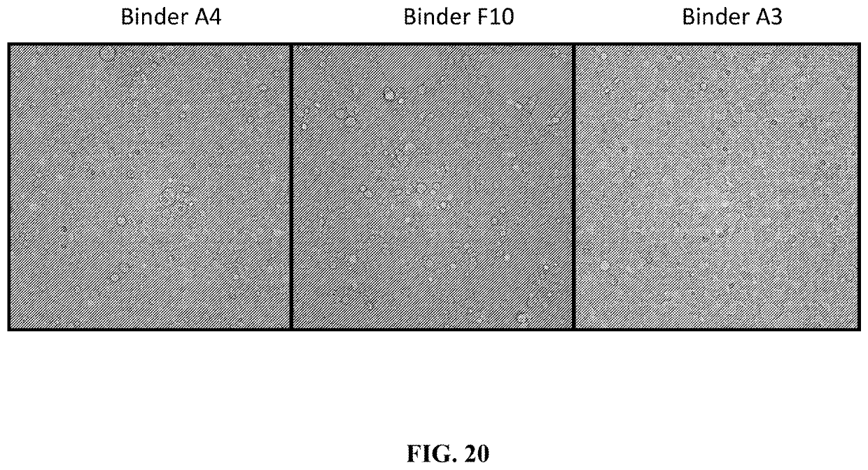

FIG. 20 shows that the A4, F10 and A3 VH domains in fusion proteins with the circularly permutated SH2 domain can be expressed in mammalian cells.

### Example 5

Cells expressing an antibody system as described herein (e.g., a B1 domain that includes a part of SHP2 that can bind to pY248 in PD-1, and a B2 domain that binds a complex formed between B1 and pY248 in PD-1), that also have a functioning PD-1 pathway are used. Control cells having a functioning PD-1 pathway, but do not express an antibody system as described herein are also used.

Recombinant PDL-1 or PDL-1 expressing cancer cells are added to both the cells expressing the antibody system, and to the control cells that do not express the antibody system. Response of the PD-1 pathway upon the addition of PDL-1 is measured.

The data will show that PDL-1-activated PD-1 pathway in the control cells that do not express the antibody system recruits the native SHP2 effector protein, and the recruited SHP2 dephosphorylates T cell effector proteins such as CD3 and ZAP70. The data will show that PDL-1-activated PD-1 pathway in the cells expressing the antibody system does not recruit SHP2, or recruit less SHP2, as compared to the cells not expressing the antibody system. Consequently, T cell effector proteins such as CD3 and ZAP70 do not get dephosphorylated, or get dephosphorylated to a lesser extent.

### Example 6

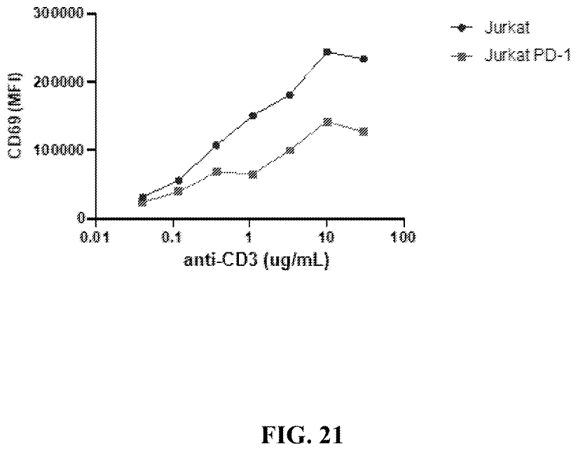

FIG. 21 shows an example of a plate-based activation assay. In the assay, cells on a plate were treated with increasing concentrations of anti-CD3 antibody, which can activate T cells. Activation of cells in response to the anti-CD3 antibody was measured using CD69 by flow cytometry. CD69 is a marker for activated T cells. The data in the figure show increasing activation of both Jurkat and Jurkat cells that express PD-1 with increasing amounts of anti-CD3 antibody. Jurkat PD-1 cells are activated less than Jurkat cells, however, due to expression of the PD-1 pathway in the Jurkat PD-1 cells.

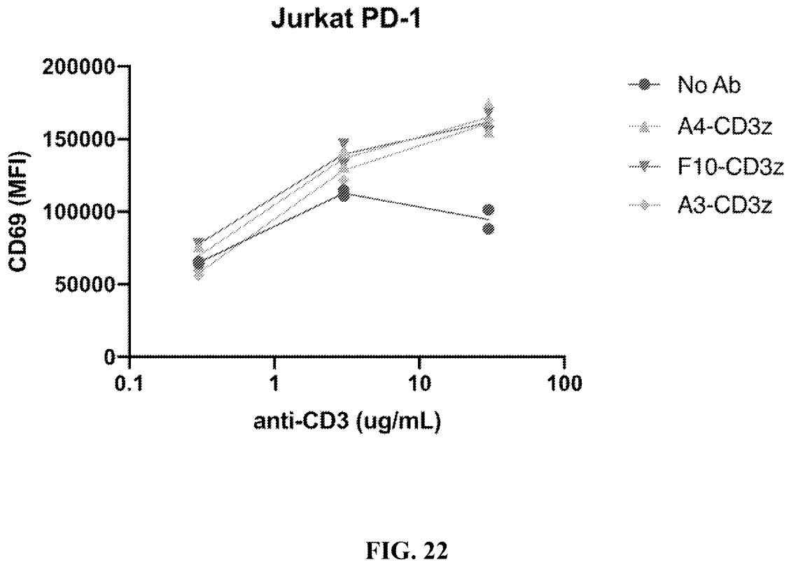

FIG. 22 shows the effects of expression of the pY-TRAP binders A4, F10, and A3 fused to a CD37 motif in Jurkat-PD1 cells, using the plate-based activation assay illustrated in FIG. 21. CD3z represents a signaling domain in CAR receptors that contains an ITAM (immunoreceptor tyrosine-based activation motif). The data indicate that the binders fused to the CD3z motif can enhance CAR-T activity. These data may indicate that the pY-TRAP binders fused to a CD3z motif can convert an immune checkpoint pathway (e.g., PD-1) to an immune activation pathway.

### Example 7

Binding of binder molecules to specific phosphorylated amino acids was tested in a model cell line system. To do this, a cell line stably expressing unphosphorylated PD-1 and, separately, a cell line stably expressing phosphorylated PD-1 were constructed.

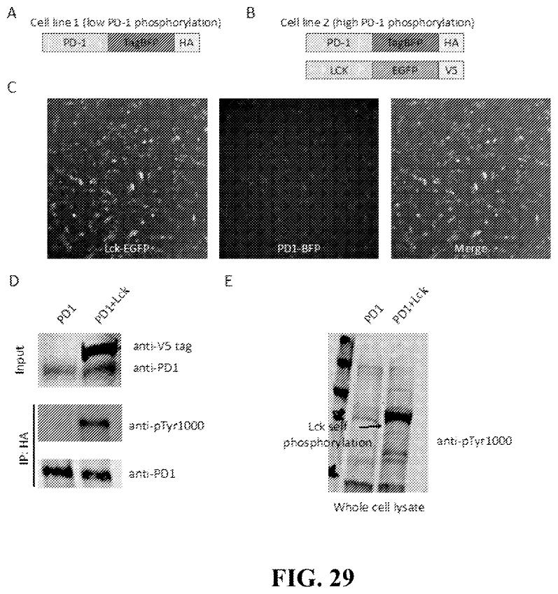

To construct the PD-1-expressing cell lines, PD1-TagBFP-HAtag (schematic shown in FIG. 29A) was stably expressed in the HEK293 cell line. PD1-TagBFP-HAtag has a PD1 molecule, a BFP fluorescent tag (blue), and an HA tag. Separately, PD1-TagBFP-HAtag and LCK-EGFP-v5tag (schematic shown in FIG. 29B) were expressed in another HEK293 cell line. LCK-EGFP-v5tag has an LCK kinase, a EGFP fluorescent tag (green), and a V5 tag. LCK is the kinase that phosphorylates PD-1. As HEK cells lack endogenous PD-1 and LCK expression, we anticipated that PD-1 would only be expressed in the HEK293 cell line also expressing the LCK kinase.

Expression and membrane localization of PD-1 and LCK in these cell lines was confirmed using fluorescence microscopy (FIG. 29C). LCK-dependent PD-1 phosphorylation was confirmed in immunoprecipitation experiments (FIGS. 29D-E).

Next, we asked whether the binder molecules could be expressed in cells and if the molecules would be functionally intact inside cells. This question was asked because some single-domain antibodies may not be natively stable in a reducing intracellular environment (e.g., due to presence of an intramolecular disulfide bond).

Multiple binder molecules were fused to an mCherry fluorescent tag (red). Among the binders tested, Binder #1, #2, #3 (F10) and #5 had moderate intracellular expression in the cells (Binder #1 and #2 are shown in FIG. 30). Other binders showed little intracellular expression (e.g., A3 as shown in FIG. 30).

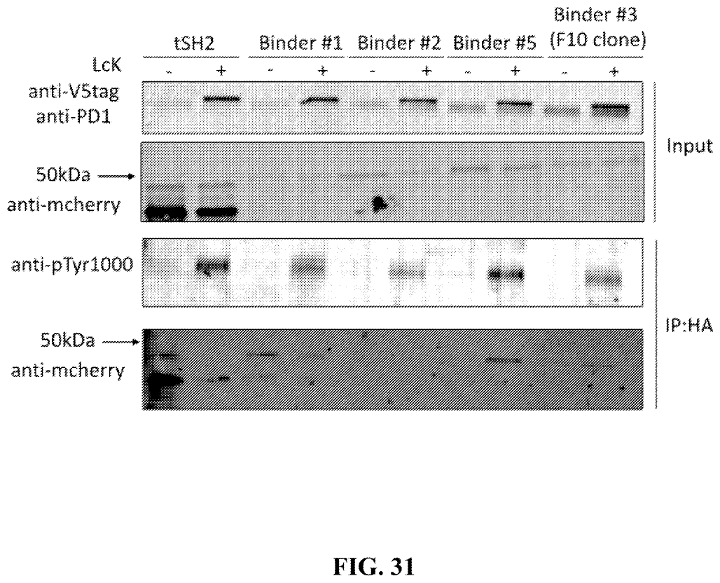

We next performed experiments to determine if intracellularly-expressed binders recognized PD-1. Various binder molecules (Binder #1, #2, #3 (F10) and #5) were expressed in both the PD-1 expressing HEK293 cells described above (unphosphorylated PD-1) and in the PD-1 and LCK overexpressing HEK293 cells (phosphorylated PD-1). Expression of PD-1 and LCK were confirmed in Western blots using anti-PD-1 and antiV5tag antibodies, respectively (Top panel in FIG. 31). Expression of binders were confirmed in Western blots using an anti-mCherry antibody (Second panel in FIG. 31). Whole-cell lysates were analyzed in the Western blots.

To test binding of the binder molecules to PD-1, immunoprecipitation experiments were performed with anti-HA beads that bind the HA-tagged PD-1-TagBFP fusion protein expressed in the cells. First, the immunoprecipitates were Western blotted with anti-pTyr1000 antibody to identify phosphorylated PD-1. Phosphorylated PD-1 was identified only in cells expressing LCK (Third panel in FIG. 31). Second, the immunoprecipitates were Western blotted with anti-mCherry antibody to identify the binder molecules. These data showed that Binders #3 and #5 were co-immunoprecipitated with PD-1 in lysates of cells expressing LCK, indicating that these binders did bind phosphorylated PD-1 intracellularly (Fourth panel in FIG. 31).

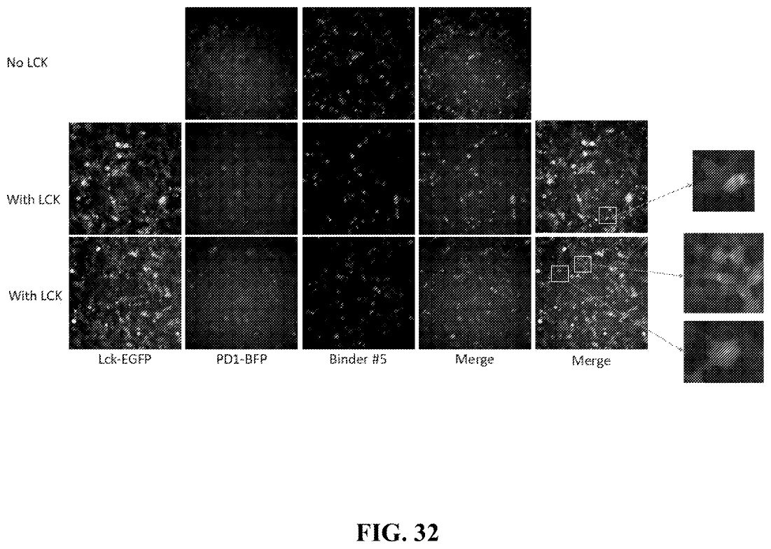

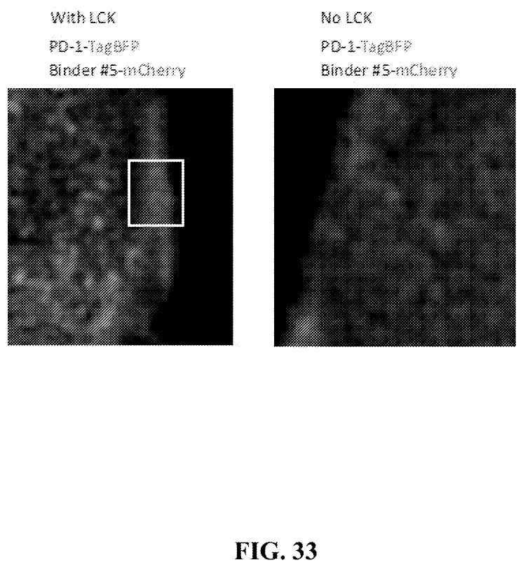

To observe localization of binder molecules inside cells, high-magnification fluorescence images were collected for cells expressing Binder #5 and either PD-1 or PD-1 and LCK. These images are shown in FIG. 32. The images show co-localization of the binder with PD-1 and LCK at the cell membrane of cells expressing PD-1 and LCK, but not in cells that lack LCK. A higher magnification of this is shown in FIG. 33.

### Example 8

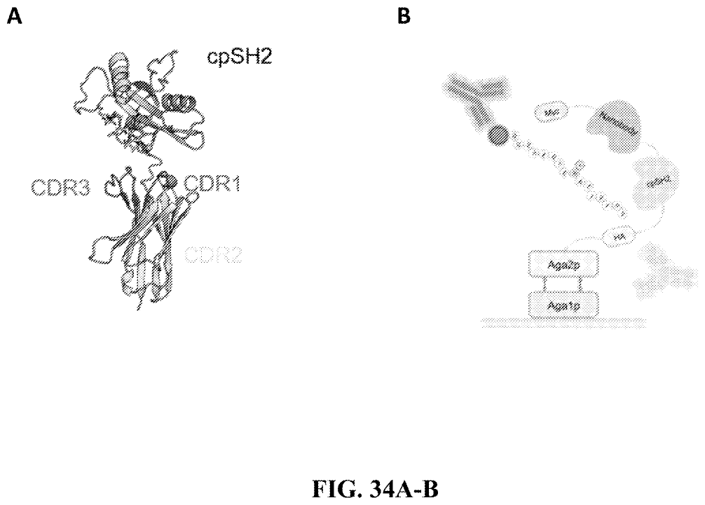

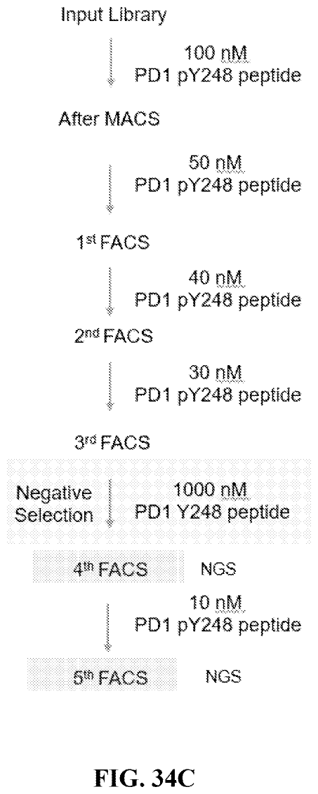

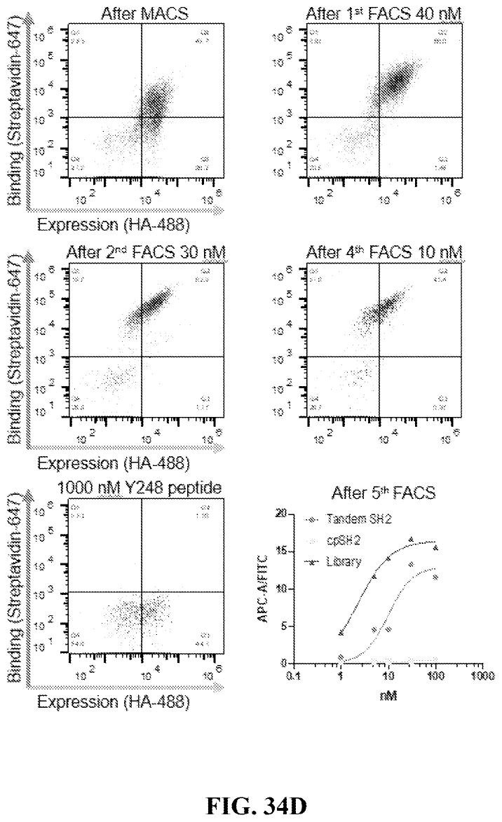

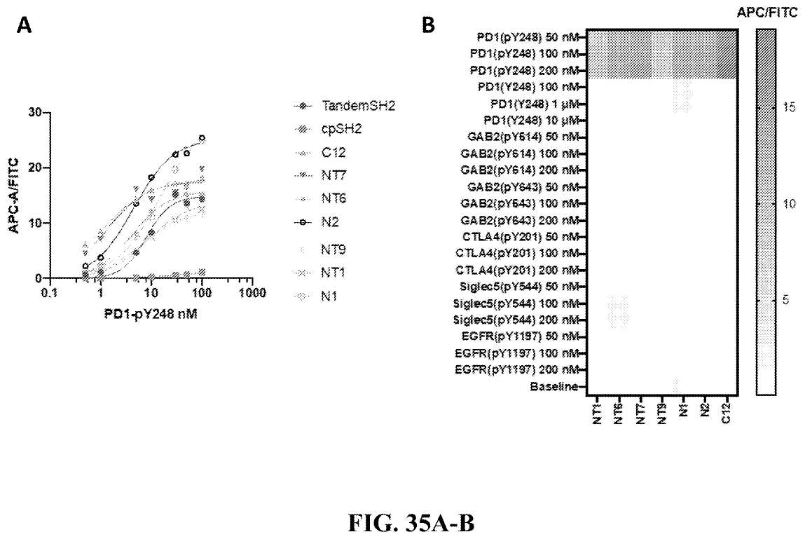

Another library was used to find binders. The molecules in this library had the circularly permuted SH2 domain (cpSH2) from SHP2 phosphatase fused to a nanobody (i.e., heavy chain) library with randomized CDR sequences (FIG. 34A; Table 6). FIG. 34B shows an example of a yeast surface display screening of a pY-TRAP in the library, tagged with an HA-tag to a biotinalyated peptide with phosphotyrosine modification (pY-peptide). FIG. 34C shows the multistep enrichment process that used both magnetic enrichment and fluorescent activated cell sorting. FIG. 34D shows flow cytometric analysis of the various enrichment steps (the populations in the upper-quadrants of the plots were enriched. A titration after the final round of enrichment is shows a nanomolar range of EC₅₀s (FIG. 35A, Table 7). These binders generally bound better than cpSH2 and tandemSH2 from SHP2.

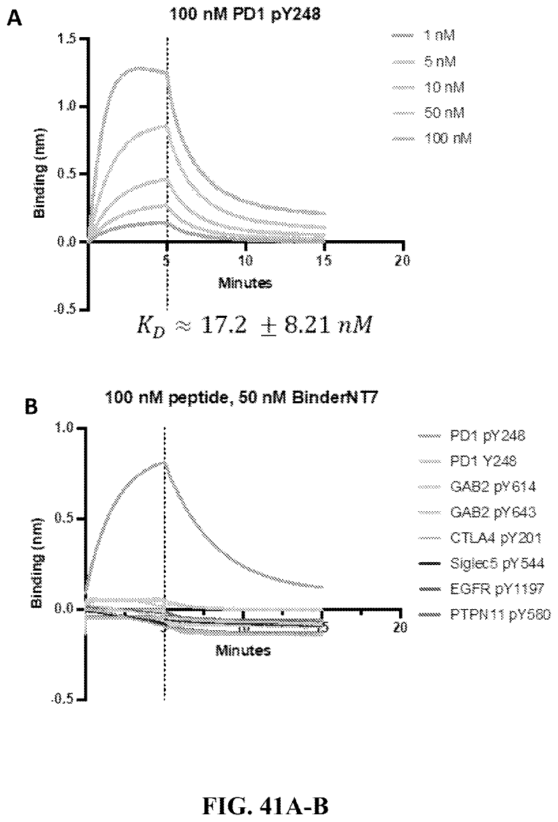

Biolayer interferometry (BLI) data indicated that the NT7 binder binds to a PD-1 pY248 peptide with a K_{D} of about 17 nm (FIG. 41A).

### Example 9

Additional studies were performed on the molecules described in Example 8 to examine their functioning.

To study binding specificity of the molecules, studies that tested binding to a panel of peptides, including unphosphorylated forms of the peptides, and pY-peptides with amino sequences similar to PD-pY248 and the endogenous binding site of the SHP2 SH2 domain were tested. The data (FIG. 35B) show binding specificity of these molecules. Biolayer interferometry of binding of the NT7 binder to a PD-1 pY248 peptide, as compared to other phosphorylated and nonphosphorylated amino acid motifs, also indicated specific binding of the molecules (FIG. 41B).

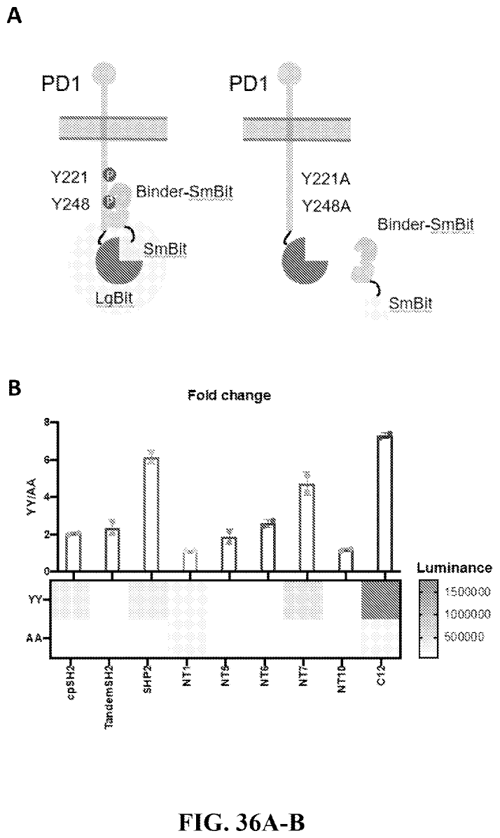

Additional studies were performed to examine binding specificity of the molecules. FIG. 36A shows a schematic diagram of a split-luciferase system where LgBit and SmBit polypeptide/peptide from luciferase combine to reconstitute a functioning luciferase molecule. As shown, wild-type PD1 (YY) or alanine mutations of both tyrosine sites (Y→A) were fused with LgBit from a luciferase. The pY-TRAP binders were fused with SmBit from the luciferase enzyme. The interaction of the Binder-SmBit and PD1-LgBit reconstitute a full luciferase, which can be measured. The data from the studies (FIG. 36B) show a heatmap of luciferase assay with cpSH2, TandemSH2 domain from SHP2, SHP2, and selected binders in LentiX cells expressing WT PD1 (YY) and mutant PD1 (AA). Fold change in luciferase signal is shown. The data show that the tested molecules have specificity for binding the phosphorylated tyrosine residues (YY).

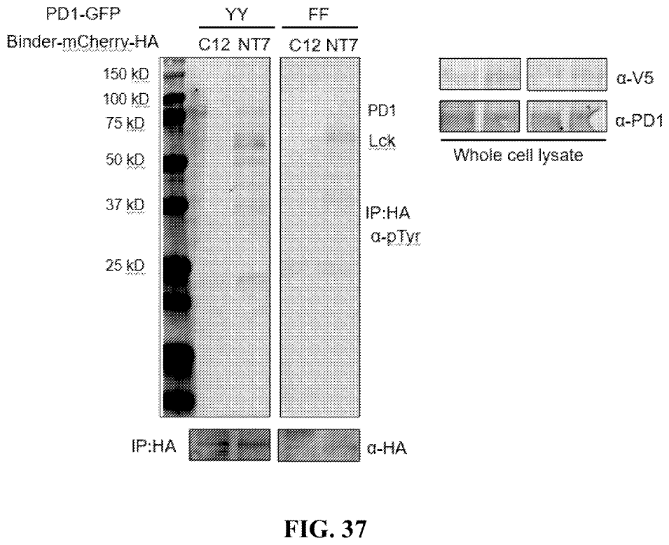

FIG. 37 shows data from studies where cell lysates from LentiX cells coexpressing Lck kinase, PD1 WT (YY) or PD1 Y223F/Y248F (FF), and selected binders tagged with mCherry and HA were immunoprecipitated with anti-HA beads (to precipitate the binders) and subsequently blotted with anti-pTyr antibody. The bands in FIG. 37 show tyrosine phosphorylation with a size corresponding to PDI and only in YY cells. Expression of Lck was detected with V5 antibody and PD1 levels were confirmed with anti-PD1 antibody in whole cell lysates. These data also show binding specificity of the tested molecules.

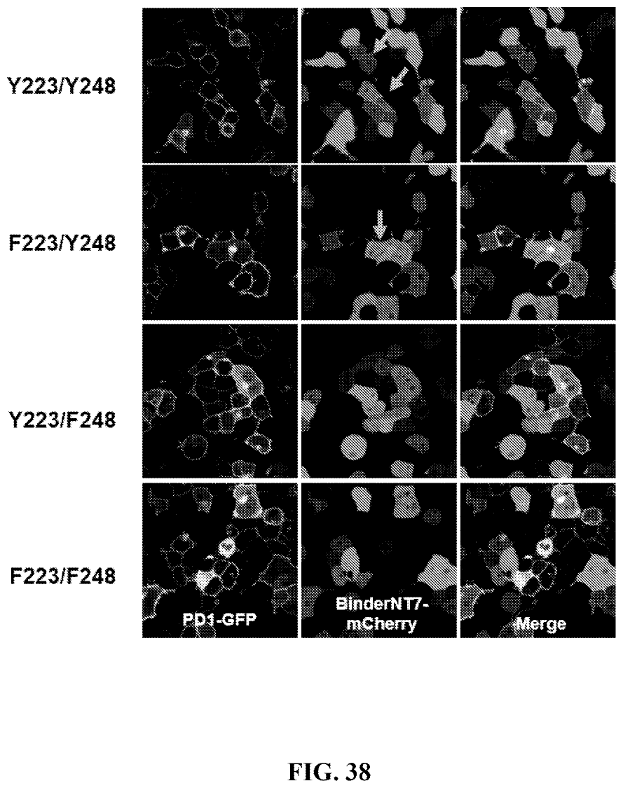

FIG. 38 shows cell fluorescence data of expression of GFP-tagged PD1 (green). The PDI molecules had either or both tyrosine residues mutated to phenylalanine. The cells used were LentiX cells co-expressing Lck and mCherry-tagged Binder-NT7 (red). Membrane localization of the binder was observed in wildtype PD1 (Y223/Y248) and F223/Y248 PD1. Membrane localization of the binder was diminished in both F223/F248 PD1 and Y223/F248 PD1.

### Example 10

Studies were performed to test whether expression of binder molecules in T cells could inhibit PDL-1-suppression of T cell activation.

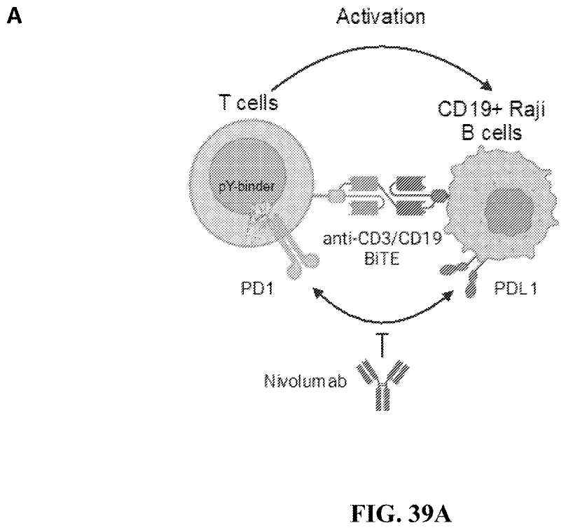

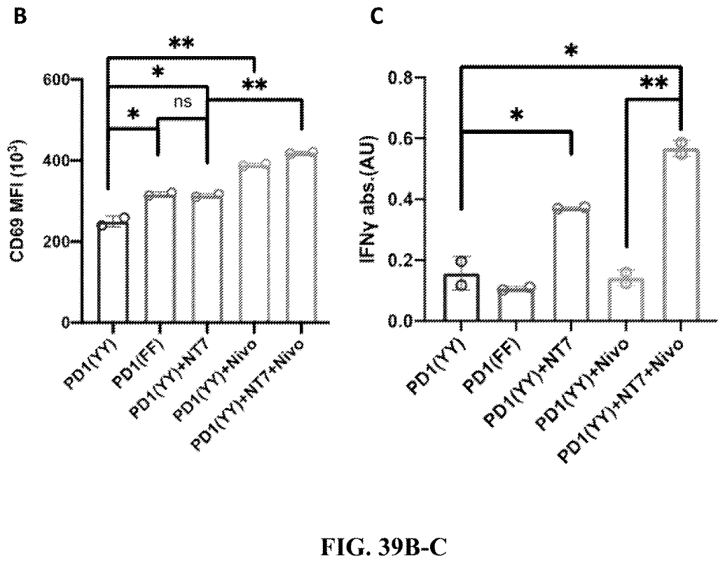

FIG. 39A is a schematic diagram showing co-culture of Raji B cells that overexpress PDL1 (right), and Jurkat T PD1−/− cells (left) reconstituted with wildtype PD1 (YY) or FF mutant PD1 (FF). Jurkat T cells were activated with 1000 μM anti-CD3/anti-CD19 bispecific antibody (BiTE).

In the data shown in FIG. 39B-C, activation of the anti-CE3/anti-CD19 antibody-treated T cells was quantified by measuring surface CD69 levels (FIG. 39B) and IFN-γ release (FIG. 39C), both indicators of T cell activation. The data showed that the tested binder molecule (NT7) reversed the PDL-1 suppression of T cell activation, as did the positive control, Nivolumab (anti-PD1). The data showed that reversal of the PDL-1 suppression by the NT7 binder plus Nivolumab was greater than reversal of PDL-1 suppression by either NT7 or Nivolumab alone.

### Example 11

A study was performed to demonstrate one way that the binder molecules described herein can be used to manipulate gene expression in cells. The study demonstrates a synthetic transcription factor can be release by a protease fused to a pY-TRAP molecule.

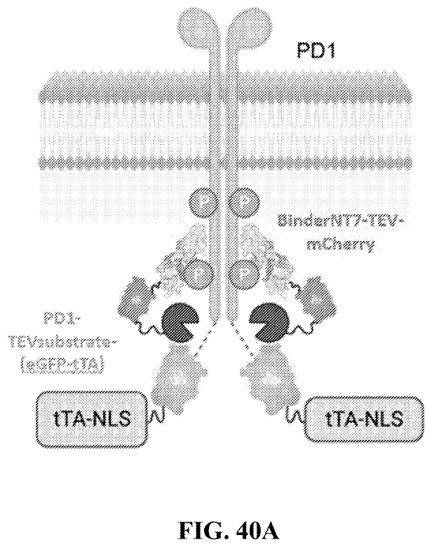

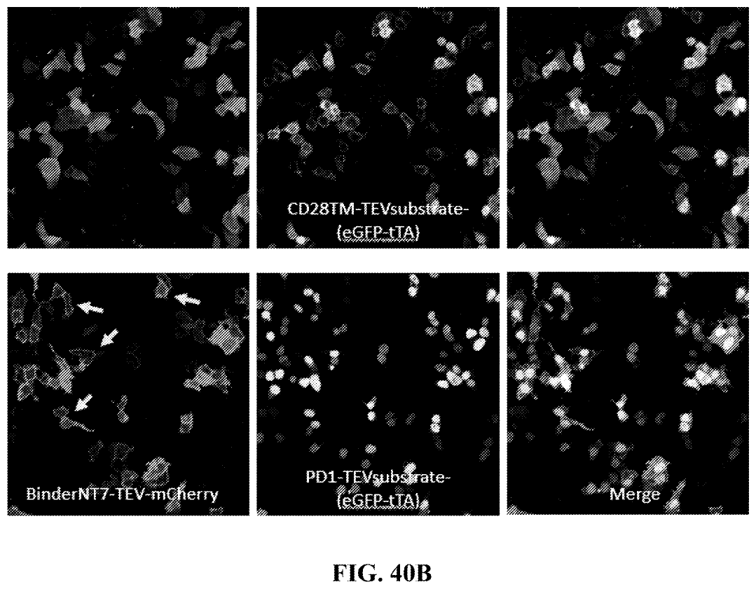

FIG. 40A is a schematic of a cellular membrane that contains a PD-1 molecule that has a tetracycline transactivator (tTA) fused to a nuclear localization signal (NLS). This molecule is called PD1-TEVsubstrate-eGFP-tTA. Not shown is a negative-control cell that has a CD28 transmembrane protein that also has a tTA fused to an NLS (called CD28TM-TEVsubstrate-eGFP-tTA0).

FIG. 40B shows data from the study. LentiX cells were co-transfected with PD1-TEVsubstrate-eGFP-tTA (bottom row) or CD28TM-TEVsubstrate-eGFP-tTA (top row), and also with BinderNT7-TEV-mCherry. In cells transfected with PD1-TEVsubstrate-eGFP-tTA, membrane recruitment of binder-fused TEV was observed (lower left) as well as nuclear localization of the eGFP-tagged tTA (lower right). No membrane recruitment of binder fused TEV in CD28TM-TEVsubstrate-eGFP-tTA cells was found (upper left), and no nuclear localization of tTA could be found (upper right).

The data show that when the binder molecule specifically binds to phosphorylated PD-1, the protease can cleave the transactivator from the PD-1 and that the cleaved transactivator can be localized to the cell nucleus because of the fused nuclear localization signal.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. An antibody system, comprising:
a first molecule (B1) that binds to a phosphorylated amino acid motif in a target protein, forming a first complex of B1 and the protein; and
a second molecule, comprising an antibody or antigen binding fragment thereof (B2) that binds to the first complex to form a second complex.

2. The antibody system of claim 1, wherein B1 comprises an antibody or antibody binding fragment thereof.

3. The antibody system of claim 1, wherein B1 comprises part of a protein that can bind to the phosphorylated amino acid motif in the target protein.

4. The antibody system of claim 3, wherein B1 comprises a protein having enzymatic activity or that can facilitate formation of a protein signaling complex.

5. The antibody system of claim 4, wherein the protein having enzymatic activity comprises an SH2 domain.

6. The antibody system of claim 5, wherein the SH2 domain is from an SHP2 protein.

7. The antibody system of claim 5, wherein the SH2 domain comprises a circular permutated SH2 domain.

8. The antibody system of claim 6, wherein the SH2 domain comprises a circular permutated C-terminal SH2 domain of SHP2.

9. The antibody system of claim 8, wherein the circular permutated C-terminal SH2 domain of SHP2 has an amino acid sequence as shown in Tables 1 and 2, or an amino sequence 90, 91, 92, 93, 94, 95, 96, 97, 98 or 99% identical thereto.

10. The antibody system of claim 1, wherein the phosphorylated amino acid motif is embedded in a three-dimensional structural domain of the target protein (tertiary folded domain of the protein).

11. The antibody system of claim 1, wherein the phosphorylated amino acid motif comprises a non-linear epitope.

12. The antibody system of claim 11, wherein the phosphorylated amino acid motif comprises a three-dimensional (folded) epitope of the target protein.

13. The antibody system of claim 1, wherein the phosphorylated amino acid motif comprises a linear epitope of the target protein.

14. The antibody system of claim 1, wherein B2 bound to the first complex binds to B1.

15. The antibody system of claim 1, wherein B2 bound to the first complex binds to the target protein.

16. The antibody system of claim 1, wherein B2 bound to the first complex binds to B1 and the target protein.

17. The antibody system of claim 1, wherein B2 bound to the first complex recognizes an epitope comprising B1.

18. The antibody system of claim 1, wherein B2 bound to the first complex recognizes an epitope comprising the target protein.

19. The antibody system of claim 1, wherein B2 bound to the first complex recognizes an epitope comprising BI and the target protein.

20. The antibody system of claim 1, wherein B1 does not bind the amino acid motif in the target protein if the amino acid motif is not phosphorylated.

21. The antibody system of claim 1, wherein B2 does not bind the first complex if B1 is not bound to the phosphorylated amino acid motif in the target protein.

22. The antibody system of claim 1, wherein the phosphorylated amino acid motif comprises a phosphohistidine, phosphoserine, phosphothreonine, or phosphotyrosine.

23. The antibody system of claim 1, wherein the phosphorylated amino acid motif is present in a signal transduction protein.

24. The antibody system of claim 23, wherein the signal transduction protein comprises a receptor.

25. The antibody system of claim 23, wherein the signal transduction protein has enzymatic activity.

26. The antibody system of claim 23, wherein the signal transduction protein regulates an immune checkpoint pathway.

27. The antibody system of claim 26, wherein the signal transduction protein comprises PD-1, CTLA-4, LAG-3, TIGIT, 2B4, BTLA, CD57, TIM-3, A2AR, VISTA, KIR, IDO, B7-H3, B7-H4, or KLRG-1.

28. The antibody system of claim 27, wherein the signal transduction protein comprises PD-1.

29. The antibody system of claim 23, wherein the signal transduction protein comprises a phosphohistidine, phosphoserine, phosphothreonine or phosphotyrosine.

30. The antibody system of claim 23, wherein a protein having enzymatic activity or that can facilitate formation of a signaling complex can bind the phosphorylated amino acid motif of the signal transduction protein.

31. The antibody system of claim 30, wherein the protein having enzymatic or complex formation ability comprises a phosphohistidine-, phosphoserine-, phosphothreonine-, or phosphotyrosine-binding domain.

32. The antibody system of claim 30, wherein the protein having enzymatic activity comprises a Src homology 2 (SH2) or phosphotyrosine-binding (PTB) domain.

33. The antibody system of claim 32, wherein the protein having enzymatic activity comprises SHP2.

34. The antibody system of claim 23, wherein the phosphorylated amino acid motif comprises a phosphotyrosine to which a protein having a Src Homology 2 (SH2) domain can bind.

35. The antibody system of claim 23, wherein the phosphorylated amino acid motif comprises a phosphoserine or phosphothreonine to which a protein having a WW domain can bind.

36. The antibody system of claim 23, wherein the phosphorylated amino acid motif comprises a phosphohistidine to which a protein having an SH2 domain can bind.

37. The antibody system of claim 23, wherein the phosphorylated amino acid motif comprises a sulfotyrosine to which a protein having a modified SH2 domain can bind.

38. The antibody system of claim 30, wherein formation of the second complex prevents a protein from binding and/or regulating the phosphorylated amino acid motif in the signal transduction protein.

39. The antibody system of claim 26, wherein formation of the second complex prevents activation of the immune checkpoint pathway.

40. The antibody system of claim 1, wherein the B1 and the B2 are covalently linked.

41. The antibody system of claim 40, wherein the B1 and or the B2 comprise an scFv, single-domain antibody, nanobody, monobody, DARPin or affibody.

42. The antibody system of claim 40, wherein the B1 and the B2 are linked with a peptide linker to form a linked B1-B2.

43. The antibody system of claim 42, wherein the linked B1-B2 additionally comprises a motif to which a phosphatase or kinase can bind.

44. The antibody system of claim 42, wherein the linked B1-B2 additionally comprises an immunoreceptor tyrosine-based activation motif (ITAM), an immunoreceptor tyrosine-based inhibitory motif (ITIM), or an immunoreceptor tyrosine-based switch motif (ITSM).

45. The antibody system of any one of claims 1-44, expressed in or introduced into a cell.

46. The antibody system of claim 45, wherein the cell comprises a T cell.

47. The antibody system of claim 46, wherein the cell comprises a CAR-T cell, a CAR-NK cell or a CAR-macrophage cell.

48. A kinase or phosphatase enzyme comprising the linked B1-B2 of claim 42.

49. A protease enzyme comprising the linked B1-B2 of claim 42.

50. The antibody system of claim 6, wherein the B2 comprises an antibody or antibody binding fragment thereof comprising a heavy chain, wherein the heavy chain comprises a CDR1, CDR2 and CDR3 as shown in Table 4 for 10.1/E4, 10.2, 10.3/F10, 10.4, 10.5, 10.6, 10.7, 30.3, 30.5, 30.7, C6, A4, B5, C8, F9, E10 or D9.

51. The antibody system of claim 50, wherein a scaffold of the heavy chain is encoded by the nucleic acid sequence as shown in Table 3, or a nucleic acid sequence 90, 91, 92, 93, 94, 95, 96, 97, 98 or 99% identical thereto.

52. The B1 of claim 1.

53. The B1 of claim 52, comprising an scFv, single-domain antibody, nanobody, monobody, DARPin or affibody.

54. The B1 of claim 52, comprising a circular permutated SH2 domain from a SHP2 protein.

55. The B1 of claim 52, having an amino acid sequence as shown in Tables 1 or 2, or an amino sequence 90, 91, 92, 93, 94, 95, 96, 97, 98 or 99% identical thereto.

56. The B1 of claim 52, wherein the BI is not covalently linked to B2.

57. The B2 antibody or antigen binding fragment thereof of claim 1.

58. The B2 of claim 57, comprising an scFv, single-domain antibody, nanobody, monobody, DARPin or affibody.

59. The B2 antibody or antigen binding fragment thereof of claim 1, comprising a heavy chain, wherein the heavy chain comprises a CDR1, CDR2 and CDR3 as shown in Table 4 for 10.1/E4, 10.2, 10.3/F10, 10.4, 10.5, 10.6, 10.7, 30.3, 30.5, 30.7, C6, A4, B5, C8, F9, E10 or D9.

60. The B2 antibody or antigen binding fragment thereof of claim 59, wherein a scaffold of the heavy chain is encoded by the nucleic acid sequence as shown in Table 3, or a nucleic acid sequence 90, 91, 92, 93, 94, 95, 96, 97, 98 or 99% identical thereto.

61. The B2 antibody or antigen binding fragment thereof of claim 57, wherein the B2 is not covalently linked to B1.

62. A bispecific antibody, comprising:
a first antigen-binding domain (B1) that binds to a phosphorylated amino acid in a signal-transduction protein; and
a second antigen-binding domain (B2) that binds to the B1 and/or the signal transduction protein when the B1 is bound to the phosphorylated amino acid in the signal-transduction protein.

63. The bispecific antibody of claim 62, wherein the B2 binds to the B1 and the signal transduction protein when the B1 is bound to the phosphorylated amino acid in the signal-transduction protein.

64. The bispecific antibody of claim 62, wherein the signal-transduction protein comprises a cellular receptor.

65. The bispecific antibody of claim 64, wherein the cellular receptor comprises an intracellular domain that includes the phosphorylated amino acid.

66. The bispecific antibody of claim 64, wherein the cellular receptor comprises a cytokine receptor.

67. The bispecific antibody of claim 64, wherein the cellular receptor can modulate cancer in a cell or in an individual.

68. The bispecific antibody of claim 62, wherein the phosphorylated amino acid is selected from the group consisting of a serine, threonine, tyrosine and histidine.

69. The bispecific antibody of claim 68, wherein the tyrosine is present in a motif to which a protein having a Src Homology 2 (SH2) domain can bind.

70. The bispecific antibody of claim 62, wherein the signal-transduction protein comprises an immune checkpoint pathway.

71. The bispecific antibody of claim 70, wherein a regulatory protein in the immune checkpoint pathway comprises PD-1, CTLA-4, LAG-3, TIGIT, 2B4, BTLA, CD57, TIM-3, KLRG-1, or a combination thereof.

72. The bispecific antibody of claim 71, wherein the signal-transduction protein comprises a PD-1 protein.

73. The bispecific antibody of claim 72, wherein the phosphorylated amino acid in the PD-1 protein comprises phosphotyrosine 223 or 248.

74. The bispecific antibody of claim 70, wherein the immune checkpoint pathway is not activated or is minimally activated when B1 binds the phosphorylated amino acid in the signal-transduction protein, and wherein B2 binds B1 and/or the signal-transduction protein.

75. The bispecific antibody of claim 62, wherein the phosphorylated amino acid in the signal-transduction protein is phosphorylated by a protein kinase.

76. The bispecific antibody of claim 75, wherein the protein kinase comprises a serine/threonine protein kinase, a tyrosine kinase or a histidine kinase.

77. The bispecific antibody of claim 76, wherein the tyrosine kinase comprises a receptor tyrosine kinase.

78. The bispecific antibody of claim 62, wherein the B2 does not bind to the B1 and/or to the signal-transduction protein if the B1 does not bind to the phosphorylated amino acid in the signal-transduction protein.

79. The bispecific antibody of claim 62, wherein the bispecific antibody comprises a peptide linker that links B1 and B2.

80. The bispecific antibody of claim 79, wherein the peptide linker is between about 3 amino acids and about 50 amino acids in length.

81. The bispecific antibody of claim 62, wherein the B1 is in a first bispecific antibody and the B2 to which the B1 binds is in a second bispecific antibody.

82. The bispecific antibody of any one of claims 62-81, expressed in or introduced into a cell.

83. The bispecific antibody of claim 82, wherein the cell comprises a T cell.

84. The bispecific antibody of claim 83, wherein the cell comprises a CAR-T cell, a CAR-NK cell or a CAR-macrophage cell.

85. A first antigen binding domain (B1) of the bispecific antibody of claim 57.

86. The B1 of claim 85, comprising an scFv, single-domain antibody, nanobody, monobody, DARPin or affibody.

87. A second antigen binding domain (B2) of the bispecific antibody of claim 57.

88. The B2 of claim 87, comprising an scFv, single-domain antibody, nanobody, monobody, DARPin or affibody.

89. A nucleic acid encoding the isolated bispecific antibody of claim 62.

90. A vector comprising the nucleic acid of claim 89.

91. A cell comprising the nucleic acid of claim 89 or the vector of claim 90.

92. The cell of claim 91, wherein the cell comprises a T cell.

93. The cell of claim 92, wherein the T cell comprises a chimeric antigen receptor (CAR)-T cell, a CAR-NK cell or a CAR-macrophage cell.

94. A method for treating a cancer, comprising administering to a patient, a CAR-T cell that expresses intracellularly, the bispecific antibody of any one of claims 62-81.

95. A method for treating a cancer, comprising administering to a patient, a CAR-NK cell or a CAR-macrophage cell, wherein the cells express intracellularly the bispecific antibody of any one of claims 62-81.

96. A method for introducing the bispecific antibody of any one of claims 62-81, or a nucleic acid encoding the bispecific antibody of any one of claims 62-81, into a cell or into a cell in an individual, comprising introducing into the cell a lipid nanoparticle comprising the bispecific antibody, or nucleic acid or a viral vector that contains the nucleic acid.

97. The method of claim 96, wherein the lipid nanoparticle contains a nucleic acid comprising mRNA.

98. A method for introducing into a cell or into a cell in an individual, the B1 and/or B2 of claim 1, comprising linking the B1 and/or B2 to a toxin or fragment thereof, or to a cell penetrating peptide to produce a toxin-linked or penetrating peptide-linked B1 and/or B2, and introducing the toxin-linked or penetrating peptide-linked B1 and/or B2 into the cell.

99. The method of claim 98, wherein the toxin or fragment thereof can bind to a receptor on a surface of the cell.

100. The method of claim 98, wherein the toxin or fragment thereof bound to the receptor can be internalized by the cell along with the B1 and/or B2.

101. The method of claim 98, wherein the penetrating peptide can be internalized by the cell along with the B1 and/or B2.

102. The bispecific antibody of any one of claims 62-81, additionally comprising a motif to which a phosphatase or kinase can bind.

103. The bispecific antibody of claim 102, wherein the motif comprises an immunoreceptor tyrosine-based activation motif (ITAM), an immunoreceptor tyrosine-based inhibitory motif (ITIM), or an immunoreceptor tyrosine-based switch motif (ITSM).

104. The bispecific antibody of any one of claims 62-81, additionally comprising a phosphatase or kinase enzyme.

105. The bispecific antibody of any one of claims 62-81, additionally comprising a protease.

106. The bispecific antibody of claim 105 wherein, when the bispecific antibody is expressed in a cell, the protease can cleave a substrate sequence of a membrane-tethered effector protein and release the effector protein from the membrane.

107. The bispecific antibody of claim 106, wherein the effector protein comprises a transcription factor, an epigenetic modulator, or an enzyme.

108. The bispecific antibody of claim 107, wherein the epigenetic modulator comprises a DNA methylase, an enzyme that modifies a histone, or a noncoding RNA.

109. A method for regulating activation of a T cell, comprising expressing in a cell, the antibody system of any one of claim 1-47 or 50-51, the kinase or phosphatase enzyme of claim 48, the protease enzyme of claim 49, or the bispecific antibody of any one of claims 62-84.

110. The antibody system of any one of claim 1-47 or 50-51, or the bispecific antibody of any one of claims 62-84, comprising a detectable marker.

111. The antibody system or bispecific antibody of claim 110, wherein the detectable marker comprises a fluorescent protein or moiety.

112. A method for detecting a specific phosphorylated amino acid in a specific protein, comprising:
contacting the antibody system of any one of claim 1-47 or 50-51, or the bispecific antibody of any one of claims 62-84 with cellular proteins in a permeabilized or viable cell, or with a cellular extract; and
detecting binding of the antibody system or the bispecific antibody to the specific phosphorylated amino acid in the specific protein.

113. The method of claim 112, wherein the detecting comprises visualizing a marker of the antibody system of bispecific antibody.

114. The method of claim 113, wherein the marker comprises a fluorescent protein or moiety.


SEQUENCE LISTING (96 sequences)

SEQ ID NO: 1 - DNA, 333 bp, synthetic construct
  source 1..333: mol_type = other DNA
gagcgtttcgattccctgactgaccttgtcgagcactacaaaaagaatccgatggttgaaacgctaggcaccgtactacagttaaagcaacccctaggtggttctgggggcggttggtttcatgggcacttgtcaggaaaagaggcagaaaagctgctgacggagaaaggaaaacacgggtcattcttggtacgtgaaagccagtcacaccccggcgactttgttctgagcgtaaggacaggtgacgataaaggcgagtccaatgatgggaagagtaaggttacgcatgtgatgattcgttgccaggagcttaagtacgatgtgggcgggggt

SEQ ID NO: 2 - protein, 111 aa, synthetic construct
ERFDSLTDLVEHYKKNPMVETLGTVLQLKQPLGGSGGGWFHGHLSGKEAEKLLTEKGKHGSFLVRESQSHPGDFVLSVRTGDDKGESNDGKSKVTHVMIRCQELKYDVGGG

SEQ ID NO: 3 - DNA, 309 bp, synthetic construct
  source 1..309: mol_type = other DNA
gaaaaatttgccacactggcggagctggttcaatactatatggaacatcacgggcagttaaaggagaagaatggtgacgtgattgaactaaagtacccgttaggaggctcaggcggtggttggtttcatcccaatatcacaggagtggaagctgaaaatctgttattaacaagaggcgtagatggcagttttctagcacgtccatccaaatctaaccccggcgattttaccttgagcgttaggaggaatggggcagtaacacatattaaaattcaaaataccggggattactacgatctttatggaggg

SEQ ID NO: 4 - protein, 103 aa, synthetic construct
EKFATLAELVQYYMEHHGQLKEKNGDVIELKYPLGGSGGGWFHPNITGVEAENLLLTRGVDGSFLARPSKSNPGDFTLSVRRNGAVTHIKIQNTGDYYDLYGG

SEQ ID NO: 5 - DNA, 378 bp, synthetic construct
  source 1..378: mol_type = other DNA
gaggttcagctggtggagtctggcggtggcctggtgcagccagggggctcactccgtttgtcctgtgcagcttctggcttcgatatttatagcagcagcgattatatcggctgggtgcgtcgcgccccgggtaagggcgaggaactggttgcacgtatctatagcagcagcggctatacctattatgccgatagcgtcaagggccgtttcactataagcgcagacacatccaaaaacacagcctacctacaaatgaacagcttaagagctgaggacactgccgtctattattgtgctcgcccgttctctttctctgttgctccgtactcttacgctattgactactggggtcaaggaaccctggtcaccgtctcctcg

SEQ ID NO: 6 - protein, 7 aa, synthetic construct
RISSYYI

SEQ ID NO: 7 - protein, 7 aa, synthetic construct
DFSYSDI

SEQ ID NO: 8 - protein, 9 aa, synthetic construct
DIYSSSDYI

SEQ ID NO: 9 - protein, 8 aa, synthetic construct
AIYSYDYI

SEQ ID NO: 10 - protein, 8 aa, synthetic construct
RFSSSSDI

SEQ ID NO: 11 - protein, 7 aa, synthetic construct
AFSYSEI

SEQ ID NO: 12 - protein, 7 aa, synthetic construct
DISSSDI

SEQ ID NO: 13 - protein, 7 aa, synthetic construct
RISSDYI

SEQ ID NO: 14 - protein, 7 aa, synthetic construct
DFSSDYI

SEQ ID NO: 15 - protein, 7 aa, synthetic construct
AFSSDSI

SEQ ID NO: 16 - protein, 8 aa, synthetic construct
RIYSSSSI

SEQ ID NO: 17 - protein, 7 aa, synthetic construct
DIYSDEI

SEQ ID NO: 18 - protein, 7 aa, synthetic construct
DFYSDYI

SEQ ID NO: 19 - protein, 7 aa, synthetic construct
FSYSDYI

SEQ ID NO: 20 - protein, 7 aa, synthetic construct
RFYYSDI

SEQ ID NO: 21 - protein, 6 aa, synthetic construct
DFSSSD

SEQ ID NO: 22 - protein, 7 aa, synthetic construct
DISSESI

SEQ ID NO: 23 - protein, 11 aa, synthetic construct
ARIYPSYGSTY

SEQ ID NO: 24 - protein, 11 aa, synthetic construct
ARIYPYYSYTY

SEQ ID NO: 25 - protein, 11 aa, synthetic construct
ARIYSSSGYTY

SEQ ID NO: 26 - protein, 11 aa, synthetic construct
ARIYPSSSYTY

SEQ ID NO: 27 - protein, 11 aa, synthetic construct
ARIYPSYGYTY

SEQ ID NO: 28 - protein, 11 aa, synthetic construct
ARIYPSYSSTY

SEQ ID NO: 29 - protein, 8 aa, synthetic construct
ARIYSSYG

SEQ ID NO: 30 - protein, 11 aa, synthetic construct
ARIYPSYNYTS

SEQ ID NO: 31 - protein, 11 aa, synthetic construct
ARIYPYYGSTY

SEQ ID NO: 32 - protein, 11 aa, synthetic construct
ARIYPYSSYTY

SEQ ID NO: 33 - protein, 11 aa, synthetic construct
ARISSYYNYTS

SEQ ID NO: 34 - protein, 11 aa, synthetic construct
ARIYPYYGYTY

SEQ ID NO: 35 - protein, 11 aa, synthetic construct
ARISPYSSYTS

SEQ ID NO: 36 - protein, 11 aa, synthetic construct
ARISSSSSYTY

SEQ ID NO: 37 - protein, 15 aa, synthetic construct
RYSFSVYPFAPSYDA

SEQ ID NO: 38 - protein, 5 aa, synthetic construct
RYGAM

SEQ ID NO: 39 - protein, 14 aa, synthetic construct
RPFSFSVAPYSYAI

SEQ ID NO: 40 - protein, 16 aa, synthetic construct
RSAPAVYYVYGNGYGM

SEQ ID NO: 41 - protein, 9 aa, synthetic construct
RYAYHVYGM

SEQ ID NO: 42 - protein, 14 aa, synthetic construct
RAVYYVPGYRVHAL

SEQ ID NO: 43 - protein, 5 aa, synthetic construct
RYSAM

SEQ ID NO: 44 - protein, 11 aa, synthetic construct
RAYAYYVDGAL

SEQ ID NO: 45 - protein, 15 aa, synthetic construct
RSYSYSVYGAAVSGL

SEQ ID NO: 46 - protein, 10 aa, synthetic construct
RAVSYYVAGL

SEQ ID NO: 47 - protein, 14 aa, synthetic construct
RSPSPSYYYVVHGM

SEQ ID NO: 48 - protein, 10 aa, synthetic construct
RYYYYVYGAM

SEQ ID NO: 49 - protein, 19 aa, synthetic construct
RFVYWVASPSDYYFVYSAF

SEQ ID NO: 50 - protein, 11 aa, synthetic construct
RSGYYWVYGAM

SEQ ID NO: 51 - protein, 15 aa, synthetic construct
RYYYSVYPVRGSSGL

SEQ ID NO: 52 - protein, 17 aa, synthetic construct
RFYYYVDGYPSYPYYAL

SEQ ID NO: 53 - protein, 23 aa, synthetic construct
QLQESGGGLVQAGGSLRLSCAAS

SEQ ID NO: 54 - protein, 11 aa, synthetic construct
GWYRQAPGKER

SEQ ID NO: 55 - protein, 36 aa, synthetic construct
ADSVKGRFTISRDNAKNTVYLQMNSLKPEDTAVYYC

SEQ ID NO: 56 - protein, 11 aa, synthetic construct
WGQGTQVTVSS

SEQ ID NO: 57 - protein, 137 aa, synthetic construct
ERFDSLTDLVEHYKKNPMVETLGTVLQLKQPLGGSGGGWFHGHLSGKEAEKLLTEKGKHGSFLVRESQSHPGDFVLSVRTGDDKGESNDGKSKVTHVMIRCQELKYDVGGGGGSQLQESGGGLVQAGGSLRLSCAAS

SEQ ID NO: 58 - protein, 9 aa, synthetic construct
GNIFNSDWM

SEQ ID NO: 59 - protein, 9 aa, synthetic construct
GYIFSRWYM

SEQ ID NO: 60 - protein, 9 aa, synthetic construct
GTIFRYEDM

SEQ ID NO: 61 - protein, 9 aa, synthetic construct
GNIFGYSYM

SEQ ID NO: 62 - protein, 9 aa, synthetic construct
GSIFGGWRM

SEQ ID NO: 63 - protein, 9 aa, synthetic construct
GYIFDAYNM

SEQ ID NO: 64 - protein, 9 aa, synthetic construct
GYISKNPVM

SEQ ID NO: 65 - protein, 9 aa, synthetic construct
GNIFGAYTM

SEQ ID NO: 66 - protein, 9 aa, synthetic construct
GYIFRTDYM

SEQ ID NO: 67 - protein, 9 aa, synthetic construct
GYISTSLDM

SEQ ID NO: 68 - protein, 9 aa, synthetic construct
GSISHSQYM

SEQ ID NO: 69 - protein, 9 aa, synthetic construct
GTIFSGWYM

SEQ ID NO: 70 - protein, 9 aa, synthetic construct
GYIFGGNIM

SEQ ID NO: 71 - protein, 14 aa, synthetic construct
ELVAGIGVGGTTNY

SEQ ID NO: 72 - protein, 14 aa, synthetic construct
EFVAGIDQGSITYY

SEQ ID NO: 73 - protein, 14 aa, synthetic construct
ELVASISYGGITYY

SEQ ID NO: 74 - protein, 14 aa, synthetic construct
EFVAAIDRGGSTYY

SEQ ID NO: 75 - protein, 14 aa, synthetic construct
EFVATITDGSTTYY

SEQ ID NO: 76 - protein, 14 aa, synthetic construct
EFVAGINAGGITYY

SEQ ID NO: 77 - protein, 14 aa, synthetic construct
ELVAGITYGTTTYY

SEQ ID NO: 78 - protein, 14 aa, synthetic construct
EFVAGINYGTSTYY

SEQ ID NO: 79 - protein, 14 aa, synthetic construct
EFVAAIDYGTNTNY

SEQ ID NO: 80 - protein, 14 aa, synthetic construct
EFVAAINSGATTYY

SEQ ID NO: 81 - protein, 14 aa, synthetic construct
EFVAGIAGGTNTNY

SEQ ID NO: 82 - protein, 14 aa, synthetic construct
EFVATIDPGGNTYY

SEQ ID NO: 83 - protein, 14 aa, synthetic construct
ELVAGINSGSNTNY

SEQ ID NO: 84 - protein, 20 aa, synthetic construct
AAYNYGAYQESVGNTLYYSY

SEQ ID NO: 85 - protein, 16 aa, synthetic construct
AAVAWAGYPWYYKHAY

SEQ ID NO: 86 - protein, 12 aa, synthetic construct
AAVFTYGSALGY

SEQ ID NO: 87 - protein, 12 aa, synthetic construct
AVLYTRGGYLGY

SEQ ID NO: 88 - protein, 12 aa, synthetic construct
AASLYSPVALAY

SEQ ID NO: 89 - protein, 12 aa, synthetic construct
AAVDDAYPPHSY

SEQ ID NO: 90 - protein, 20 aa, synthetic construct
AVGPIYPGYIDERFGYDYQY

SEQ ID NO: 91 - protein, 17 aa, synthetic construct
AAVDDYSFILGSSTHLY

SEQ ID NO: 92 - protein, 20 aa, synthetic construct
AAYYDYDWDRPENTYGYFRY

SEQ ID NO: 93 - protein, 12 aa, synthetic construct
AAWSRPSYTLSY

SEQ ID NO: 94 - protein, 16 aa, synthetic construct
AVPGYGRYQRTSVFKY

SEQ ID NO: 95 - protein, 16 aa, synthetic construct
AARYQDSHADGFTLGY

SEQ ID NO: 96 - protein, 12 aa, synthetic construct
AVSYSEGGPFYY